(19) United States
(12) Patent Application Publication
(10) Pub. No.: US 2023/0105253 A1
(43) Pub. Date: Apr. 6, 2023

(54) USE AND METHOD FOR REDUCING THE VIRAL, BACTERIAL AND FUNGAL SPORE LOAD OR OTHER BIOLOGICAL CONTAMINANTS IN GASES

(71) Applicant: MECADI GMBH - CHEMICALS/ PROCESSING, Bexbach (DE)

(72) Inventors: Andreas KONRAD, Blleskastel (DE); Wolfgang SIPOS, Mannheim (DE)

(21) Appl. No.: 17/907,192

(22) PCT Filed: Mar. 24, 2021
(86) PCT No.: PCT/EP2021/057598

§ 371 Date: Sep. 23, 2022

(30) Foreign Application Priority Data
Mar. 24, 2020 (DE) 10 2020 001 896.3
Jan. 20, 2021 (DE) 10 2021 101 099.3

(51) Int. Cl. A61L 9/014; A61L 9/20; A61L 9/22; A61L 9/14

(52) U.S. Cl. CPC A61L 9/014; A61L 9/20; A61L 9/22; A61L 9/145; A61L 2209/22; A61L 2209/14

(57) ABSTRACT

The present invention relates to the use of an ion exchanger for the removal and/or reduction of biological contaminants, such as viruses, bacteria and fungal spores, in gases and gas streams, such as room air and breathing air, and a corresponding method. In certain embodiments, the ion exchanger is a cation exchanger partially or substantially completely loaded with H⁺ ions or an anion exchanger partially or substantially completely loaded with OH⁻ ions. Additionally or alternatively, the ion exchanger may be loaded with transition metal ions, such as titanium, copper and/or silver ions.

The present invention relates to the use of an ion exchanger for the removal and/or reduction of biological contaminants in gases and/or gas streams, and a corresponding method.

Germs, viruses and other potentially harmful biological contaminants in air are present in widely varying particle sizes. The dimensions range from about 10 nm for individual non-enveloped viruses to several micrometers for particles such as agglomerates, adherents to dust or pollen, or aerosols, which contain liquids, especially water. The selective retention of such particles, especially by filtration, requires separation media with correspondingly small pore sizes. However, the smaller the pores, the higher the pressure drop along the separation medium. The achievable filter throughput per surface area becomes lower, which increases the energy input. Filters can be further reduced in their throughput by larger dust particles and thus require pre-filter systems.

The efficient reduction of biological contaminants from air, in particular breathing air, contributes to the reduction of the spread of airborne diseases. In particular, pathogens that affect the upper respiratory tract and are spread in the ambient air by exhalation, in the form of aerosols, and by speaking but especially by coughing and/or sneezing, in the form of droplets, such as influenza viruses and coronaviruses, should be mentioned here. Exemplarily, the following pathogens are mentioned, to which the staff of medical facilities, especially in the field of ENT, is exposed: Staphylococcus spp. Streptococcus spp., hepatitis B virus, human immunodeficiency virus (HIV), cytomegalovirus (CMV), herpes simplex virus (HSV-1, HSV-11) and human T-lymphotropic virus (HTLV-11/LAV).

The situation is further complicated by the increasing prevalence of antibiotic-resistant, in some cases multi-resistant bacteria, such as methicillin-resistant Staphylococcus aureus (MRSA), Pseudomonas spec., Escherichia coli arcB and Mycobacterium tuberculosis.

Plasmid DNA or free DNA with resistance properties as mobile genetic elements can also lead to the spread or transfer of resistance properties by horizontal transfer mechanisms such as conjugation, transformation or transduction. In this respect, the removal of such fragments is of considerable importance, especially in areas such as medical facilities, production facilities for antibiotics, or animal breeding.

These fragments are typically particularly small (about 30 nm in diameter, about 600 kD (kilodaltons) and require appropriate pore sizes when using filters acting via size exclusion. It is also known that plasmids can cross barriers by elongation, which further reduces the required pore size.

Other biological contaminants, such as proteins and pollen, can cause allergic reactions and asthma. Moulds and fungal spores can contain or release toxins that can cause a range of health problems. Air containing biological contaminants can be widely distributed through indoor building ventilation systems. Therefore, it is necessary to remove and/or reduce the amount of biological contaminants from these air volumes. Prior art approaches for removing and/or reducing biological contaminants are known.

CN 205 760 203 U describes a system for purifying air from pathogens, wherein the filter is described as a combination of a HEPA-9 filter, a salt filter, a nano-microfiber filter and an activated carbon filter. The system is said to kill various pathogens due to the high salt content, so that they do not colonize the HEPA filter and reduce its efficiency. However, in principle, pathogens may colonize the filters and put the user at risk when changing filters. The system is complex to set up and HEPA filtration also requires a lot of energy input.

CN 111 271 790 A discloses a device that purifies air from virus aerosols. The device comprises an array of 10 μm and 1 μm prefilters, a 0.3 μm fine filter, as well as a UV source for sterilization, an ultrasonic generator, a high-voltage electrostatic sterilization device and the use of components made of copper. The device is very complex and scale-up to large air volumes is difficult.

WO 2018/033793 A1 describes materials and devices for the deactivation of pathogens in aerosols. As a solution, a salt finishing of surfaces is proposed, so that upon sorption of aerosols, viruses and bacteria die due to a high osmotic pressure in the solution. The disadvantage of this invention is that the salt finish of surfaces or fibres is water-soluble, adhesion in the dry state is limited, especially to plastics, and the salts may be released and thus also lose their effectiveness.

In WO 98/57672 A2 a method for the removal of viral and molecular pathogens from liquid biological material by means of ion exchangers is described. In accordance with the invention, diethylaminoethyl-functionalized anion exchangers in the pH range from 5.2 to 7.2 are preferably used for this purpose.

GB 1,092,754 A discloses the removal of viruses from blood using a mixture of anion and cation exchangers having particle sizes between 297 and 1190 μm. The use of the sodium, potassium and chloride forms of the exchangers is described.

The problem underlying the present invention is thus the efficient removal and/or reduction of biological contaminants from gases, in particular air, and gas streams. In addition, the removal and/or reduction should take place with low pressure loss. The system used should also be easily recyclable, have a low environmental impact, pose no risk of contamination to the technician during replacement and should be limited as little as possible in its scope of use. The system should be long-term stable, preferably over a wide temperature range.

One or more of the foregoing problems are solved by the present invention. Surprisingly, it has been found that ion exchangers, in particular cation exchangers in the H⁺ form, anion exchangers in the OH⁻ form, cation exchangers loaded with transition metal ions, ion exchangers bearing chelating ligands, and mixtures thereof, exhibit particularly high efficiency in the filtration or removal of biological contaminants from gases, in particular air, and/or gas streams. Surprisingly, cation exchangers in the H⁺ form and anion exchangers in the OH⁻ form are particularly efficient.

According to a first aspect, the present invention thus relates to the use of an ion exchanger for removing and/or reducing biological contaminants in gases and/or gas streams. Another aspect of the present invention relates to a method for removing and/or reducing biological contaminants in gases and/or gas streams, characterized in that the gases and/or gas streams are passed through an ion exchanger.

In a preferred embodiment of the present invention, the biological contaminants are selected from the group consisting of viruses, bacteria, molds, fungal spores, mites, pollen, and fragments of the foregoing, metabolites such as mycotoxins, proteins, RNA and DNA, preferably selected from the group consisting of enveloped viruses, non-enveloped viruses, bacteria, fungal spores and proteins and particularly preferably selected from the group consisting of coronaviruses, SARS-type viruses, SARS-CoV-2 viruses, resistant pathogens and multi-resistant pathogens. The terms “SARS-type viruses” and “SARS-CoV-2 viruses” also include mutations of said viruses.

In another preferred embodiment of the present invention, the ion exchanger according to the invention comprises at least one cation exchanger, wherein the at least one cation exchanger may be a weakly acidic and/or a strongly acidic cation exchanger, and preferably is a strongly acidic cation exchanger. The cation exchanger may be partially or substantially completely loaded with H⁺ ions.

In still another preferred embodiment of the present invention, the ion exchanger is selected from the group consisting of anion exchangers, mixed anion and cation exchangers, cation exchangers loaded with transition metal ions, ion exchangers bearing chelate ligands, mixtures thereof, and mixtures thereof with cation exchangers. Preferably, the anion exchanger is partially or substantially completely loaded with OH⁻ ions.

In still another preferred embodiment of the present invention, the ion exchanger according to the invention comprises at least one cation exchanger loaded with transition metal ions, wherein the transition metal ions are selected from the group consisting of Ti, V, Cr, Mo, W, Mn, Fe, Ru, Co, Ni, Pd, Pt, Cu, Ag, Au, Zn, Cd, Hg, Pb, Ge, Ce, Sn, Al, a lanthanide, and mixtures thereof, and preferably selected from the group consisting of copper, silver, titanium, and mixtures thereof.

In another preferred embodiment of the present invention, the ion exchanger further comprises hygroscopic auxiliary agents and/or hygroscopic functional groups.

In still another preferred embodiment of the aspects of the present invention, the gas to be filtered is air, preferably selected from the group consisting of room air and breathing air. According to the present invention, the room air is preferably selected from the group consisting of room air in shelters, motor vehicle interiors, air-conditioned rooms, cabins, airplanes, emergency vehicles, truck cabs, vehicles of security forces, hospital rooms, in particular intensive care units, staff rooms, rooms for animal husbandry and other rooms in which people, animals or plants are present or located.

In a preferred embodiment of the present invention, the ion exchanger is designed as a filling of a hollow body and/or as a porous molded body. Preferably, the hollow body and/or the molded body is inserted or incorporated into or connected to a respiratory mask. In another preferred embodiment, the ion exchanger is provided as a filling of a hollow body, which can be integrated into or installed in air circulation systems, air conditioning systems, ventilation systems, exhaust systems and cleaning devices.

In still another preferred embodiment of the present invention, one or more methods selected from the group consisting of filtration, humidification, drying, condensation, UV treatment, treatment with corona or plasma, treatment with high voltage, treatment with radioactive radiation, treatment by heating, treatment by cooling, ozonization, dosing of gases or liquids for treatment of the ion exchanger and/or the gas, in particular air, is carried out additionally. The treatment may be carried out permanently or at intervals. According to the present invention, the method or use according to the invention can be combined with one or more of the aforementioned methods in order to obtain an even more efficient or effective decontamination.

Generally, it is customary to express the reduction of germs by the log reduction value (log steps); a log reduction value corresponds to a power of ten. Combining the method or use according to the invention with one or more of the aforementioned method steps preferably leads to an increase in the log reduction value, to an extension of the service life, and/or to a broader effectiveness against different types of pathogens, or optionally to an emergency filter function in the event of failure of a process step. With respect to the method steps or stages that may be used in combination:

Filtration steps may, for example, protect a packing of ion exchange resins according to the invention from blocking by coarse dust and/or may themselves effect further or preliminary reduction of pathogens.

Humidification and drying can provide improved or optimum conditions for the ion exchanger according to the invention. This includes the prevention of complete desiccation and the formation of condensates which lead to an increase in pressure drop. The condensation of liquid itself can additionally reduce aerosols. UV treatment can additionally kill pathogens in the air stream, or pathogens that are on the surface or between ion exchange particles of the invention. Ion exchangers are electrically conductive. In this respect, the ion exchangers themselves or the ion exchangers in an electrolyte can conduct electric current. Thus, the effectiveness of the ion exchangers themselves against pathogens can be increased or continuous regeneration can take place, in particular through the formation of H⁺ and OH⁻-ions at electrodes. By combination with high voltage, corona or plasma, pathogens can be additionally reduced in the gas streams by creating active species such as radicals or ozone. Treatment with radioactive radiation is a known method for reducing pathogens, which can be combined with the use of ion exchangers according to the invention. Heat can be applied both to reduce the number of pathogens in air streams in combination with the ion exchanger. However, the ion exchanger can also be heated to cause pathogen reduction or sterilization of the exchanger or accelerated release of effective ions. Mass transfer of ions effective against pathogens from the interior of ion exchangers according to the invention to the surface to pathogens located thereon often proceeds more rapidly at higher temperatures, allowing shorter contact times and higher flow-through of an ion exchanger-filled assembly. As an additional method step, cooling can result in condensation of liquids and additional reduction of aerosols. As an additional method step, before or after treatment of the air stream with an ion exchanger, an even further reduction of pathogens can be effected by dosing gases or liquids known to be effective against pathogens. The dosing may also be done directly into, for example, a packing of the ion exchanger, in which case the exchanger may also receive the gases or liquids. The addition of water as vapor or as liquid may also serve to prevent the exchanger from drying out. Metering acids into cation exchangers or bases into anion exchangers may also result in regeneration of the exchanger.

For the purposes of this application, the following terms have the following meanings:

According to the IUPAC Recommendations, the term “ion exchanger” means a solid or liquid inorganic or organic compound containing ions which can be exchanged with other ions of the same charge sign present in a solution in which the ion exchanger is considered insoluble. Accordingly, a “cation exchanger” refers to an ion exchanger that contains cation-loaded anionic functional groups and exchanges cations, and an “anion exchanger” refers to an ion exchanger that contains anion-loaded cationic functional groups and exchanges anions. The ion exchangers of the present invention are solids, particularly resins or gels.

“Biological contamination” is understood to mean impurities of biological origin or biological nature, which may be present in gases and/or gas streams in isolated form or in the form of agglomerates, adherents to dust or pollen or aerosols, and which are not low molecular weight compounds. A “low molecular weight compound” is considered to be a compound that does not exceed a molecular weight of 200 Da, preferably 750 Da and particularly preferably 1000 Da. In particular, the term “biological contamination” includes bacteria, viruses, molds, fungal spores, mites, pollen, as well as fragments of the foregoing, metabolic products, such as mycotoxins, proteins, RNA and DNA, for example plasmid DNA or free DNA.

The term pair “removal and/or reduction” of biological contaminants means that the amount of biological contaminants in the gases and gas streams is reduced when the ion exchanger is contacted with the gases and gas streams, i.e., gas entering the ion exchanger has a higher loading of biological contaminants than gas exiting therefrom. In a preferred embodiment, the gas or gas stream is passed through the ion exchanger and the exiting gas contains a lower amount of biological contaminants compared to the injected gas or gas stream, preferably an undetectably low amount. The amount of biological contamination can be determined via methods known to the person skilled in the art, for example microbiologically, molecular biologically or by MALDI-TOF-MS or HPLC. The removal and/or reduction of specifically bacteria and viruses can be assessed according to EN 14126, analogous to ISO 16603 and ISO 22610.

The use of the ion exchanger in “gases and/or gas streams” means that the ion exchanger can be used both to remove and/or reduce biological contaminants in substantially static, i.e., non-flowing, gases, and to remove biological contaminants in flowing gases. In the former case, the ion exchanger may be actively agitated in the gas to be contacted with as much of the gas as possible. In the second case, the flowing gas is actively moved through the ion exchanger, for example by fans or blowers. In this context, gas and/or gas stream are understood to be pure gases (e.g. nitrogen, oxygen and helium), gas mixtures (e.g. air, in particular room air, or synthetic air), and dispersions of solid or liquid substances with gases or gas mixtures (e.g. aerosols, mist, smoke, dust and fine dust).

An “aerosol” is understood to be a dispersion of solid or liquid substances with gases or gas mixtures, in which at least 50% by weight of the particles have a particle size of less than 10 μm, preferably less than 5 μm. The particles have predominantly a size between 0.1 μm to 10 μm or 0.1 to 5 μm, the smallest particles are a few nanometers in size, there is no upper limit. However, very large particles sediment very quickly and are therefore of little practical relevance.

### The Ion Exchanger

The ion exchanger of the present invention may be a cation exchanger, an anion exchanger, a mixed anion and cation exchanger, a cation exchanger loaded with transition metal ions, an ion exchanger bearing chelating ligands, or a mixture thereof. Particularly preferably according to the present invention, the ion exchanger is an “organic ion exchanger”. Organic ion exchangers according to the present invention are based on polymers or copolymers containing anionically and/or cationically charged functional groups and are preferably cross-linked. Organic ion exchangers include so-called “synthetic resin ion exchangers” and are also referred to herein as “polymer-based ion exchangers”.

If pKs values are given below for functional groups of the ion exchangers, these serve to define or classify the respective group and in no way describe the pH value in the application according to the invention.

In a preferred embodiment of the present invention, the ion exchanger is a cation exchanger. The cation exchanger may be an inorganic or organic cation exchanger. Preferably, the cation exchanger is an organic cation exchanger. Organic cation exchangers, in particular cationic synthetic resin ion exchangers, are based on preferably cross-linked polymers or copolymers containing anionically charged functional groups.

Examples of typical crosslinkers are divinylbenzene, trivinylbenzene, ethylene diacrylate, diallyl maleate or diallyl fumarate or diallyl itaconate.

Polyunsaturated polymerizable substances such as divinylbenzene, ethylene dimethacrylate, diallyl ether or allyl acrylate can also be copolymerized with acrylic or methacrylic acid. The polymerization is carried out, for example, with peroxides such as benzoyl peroxide. The polymerization may be carried out, for example, as an emulsion polymerization. In this case, the skilled person can control the particle size distribution of the resulting ion exchangers by controlling the polymerization conditions.

As a polymeric base of the ion exchangers, biopolymers are also possible as carriers, such as sepharose, agarose or cellulose.

In one embodiment of the present invention, the at least one cation exchanger is a weakly acidic cation exchanger and/or a strongly acidic cation exchanger.

A “strongly acidic cation exchanger” is understood to be a cation exchanger containing anionically charged functional groups, wherein the free acid of these anionically charged functional groups has a pKs value of 3 or less, preferably 2 or less. In the case of acids having multiple dissociation states and consequently multiple pKs values, this requirement is satisfied if one of the pKs values is within the specified range. Sulfonate groups are an example thereof. In one embodiment, the strongly acidic cation exchanger is a cross-linked polystyrene sulfonate or cross-linked poly(2-acrylamido-2-methyl-1-propanesulfonic acid) (polyAMPS). Another embodiment concerns formaldehyde condensates containing methyl sulfonic acid groups, sulfonated zeolites, sulfonated activated carbons, sulfonated lignites, or sulfonated cross-linked lignins. Other embodiments include sulfonated natural materials or generally sulfonated materials in the form of fibers, ground materials, or molded bodies. Cation exchangers having other strongly acidic groups covalently bonded to a macromolecular or inorganic structure are also “strongly acidic cation exchangers” within the meaning of the present invention. The strongly acidic cation exchanger may also contain a plurality of different anionically charged functional groups, wherein the free acid of each of these anionically charged functional groups has a pKs value of 3 or less. Exemplary strongly acidic cation exchangers provided in accordance with the invention are Dowex® 50W-X8 (strongly acidic cation exchanger in the H⁺ form) which contains sulfonic acid groups in a styrene matrix crosslinked with divinylbenzene. Other examples of commercial exchangers include Amberlite® IR 120, Amberlite® IRC 120, the macroporous Amberlyst® 14 and Dowex 50 \A/X-4.

A “weakly acidic cation exchanger” is understood to be a cation exchanger containing anionically charged functional groups, wherein the free acid of these anionically charged functional groups has a pKs value in the range of more than 3 up to and including 7, preferably more than 3 up to and including 5. Carboxylate or carboxylic acid groups are an example thereof. In one embodiment, the weakly acidic cation exchanger is a cross-linked polyacrylate, a polymer of maleic anhydride and styrene, or a copolymer of divinylbenzene and acrylic or methacrylic monomers. The weakly acidic cation exchanger may also comprise a plurality of different anionically charged functional groups, wherein the free acid of each of these anionically charged functional groups has a pKs value of from greater than 3 up to and including 7. Examples of commercially available weakly acidic cation exchangers provided in accordance with the invention include Serdolit CW-1 (Serve), Ion Exchange IV (Merck) and Resinex K-H.

Cation exchangers containing different anionically charged functional groups, the free acid of one part of said anionically charged functional groups having a pKs value in the range of 3 or less and the free acid of another part of these anionically charged functional groups having a pKs value in the range of more than 3 up to and including 7, are referred to herein as “medium acid cation exchangers”.

In a particularly preferred embodiment of the present invention, the cation exchanger is partially or substantially completely loaded with H⁺ ions. By a cation exchanger partially loaded with H⁺ ions is meant a cation exchanger in which at least 50 mol %, preferably at least 75 mol % and particularly preferably at least 80 mol % of all anionically charged functional groups are present in the protonated form, i.e., in the form of their free acid. By a cation exchanger substantially completely loaded with H⁺ ions is meant a cation exchanger in which at least 90 mol-%, preferably at least 95 mol-% and particularly preferably at least 98 mol-% of all anionically charged functional groups are present in the protonated form, i.e., in the form of their free acid. The remaining functional groups carry cations as counterions. According to one embodiment, these may be selected from the group consisting of alkali metal ions, alkaline earth metal ions, ammonium or phosphonium, transition metal cations and mixtures thereof. Commercially available cation exchangers usually contain alkali metal ions and can be converted to the H⁺ ion form by washing with acids. The cation exchanger partially or substantially completely loaded with H⁺ ions thus obtained thus usually still contains certain amounts of alkali metal ions or other cations.

The strongly acidic cation exchanger partially or substantially completely loaded with H⁺ ions can be obtained by washing the cation exchanger with an aqueous solution of a strong acid. By a “strong acid” is meant an acid having a pKs value of 2 or less, preferably 0 or less. The pKs value can be obtained from commonly used textbooks. The strong acid may be selected from the group consisting of hydrochloric acid, sulfuric acid, hydrobromic acid, nitric acid, phosphoric acid and mixtures thereof. The aqueous solution preferably has a pH in the range of from 0 to 2, preferably from 0 to 1.5, and more preferably from 0.5 to 1.

In a preferred embodiment, the remaining functional groups bear transition metal cations selected from the group consisting of cations of Ti, V, Cr, Mo, W, Mn, Fe, Ru, Co, Ni, Pd, Pt, Cu, Ag, Au, Zn, Cd, Hg, Pb, Ge, Ce, Sn, Al, a lanthanide and mixtures thereof, preferably selected from cations of the group consisting of Cu, Ag, Ti and mixtures thereof. The ions may be present in various oxidation states. In particular, iron may be present as Fe⁺⁺ and/or Fe⁺⁺⁺, copper may be present as Cu⁺ and/or Cu²⁺, and titanium may be present as Ti⁺⁺, Ti⁺⁺⁺ and/or Ti⁺⁺⁺⁺. Particularly preferably, the cations are selected from the group consisting of Cu⁺, Cu²⁺, Ag⁺, Ti⁺⁺⁺⁺ and mixtures thereof.

Alternatively, the cation exchanger may be partially or substantially completely loaded with transition metal cations selected from the group consisting of cations of Ti, V, Cr, Mo, W, Mn, Fe, Ru, Co, Ni, Pd, Pt, Cu, Ag, Au, Zn, Cd, Hg, Pb, Ge, Ce, Sn, Al, a lanthanide, and mixtures thereof, preferably selected from the group consisting of cations of Ti, Cu, Ag, and mixtures thereof, and particularly preferably selected from the group consisting of Cu⁺, Cu²⁺, Ag⁺, Ti⁺⁺⁺⁺, and mixtures thereof. By a cation exchanger partially loaded with transition metal cations is meant a cation exchanger in which at least 50 mol %, preferably at least 75 mol %, and particularly preferably at least 80 mol % of all anionically charged functional groups are electrically neutralized with a transition metal cation. A cation exchanger substantially completely loaded with transition metal cations is understood to mean a cation exchanger in which at least 90 mol %, preferably at least 95 mol % and particularly preferably at least 98 mol % of all anionically charged functional groups are electrically neutralized with a transition metal cation.

The loading with the transition metal cations can be accomplished by applying a solution of a transition metal ion salt, such as a transition metal ion chloride, bromide, sulfate, nitrate, perchlorate, acetate, or trifluoroacetate, to the cation exchanger substantially completely loaded with H⁺ ions. The loading obtained depends on the amount used as well as on factors such as selectivity, residence time, temperature, pH and solubility. High loadings are achieved in columns in continuous flow according to methods known to the skilled person. To ensure complete loading, an excess of the transition metal ion salt is usually required.

In a preferred embodiment of the present invention, the cation exchanger is not loaded with transition metal cations selected from the group consisting of Cu, Ag, and mixtures thereof. Particularly preferably, the cation exchanger is not loaded with transition metal cations selected from the group consisting of cations of Ti, V, Cr, Mo, W, Mn, Fe, Ru, Co, Ni, Pd, Pt, Cu, Ag, Au, Zn, Cd, Hg, Pb, Ge, Ce, Sn, Al, a lanthanide, and mixtures thereof. In this embodiment, the cation exchanger is, in other words, substantially free of transition metal cations selected from the group consisting of Cu, Ag, and mixtures thereof, and preferably substantially free of transition metal cations selected from the group consisting of Ti, V, Cr, Mo, W, Mn, Fe, Ru, Co, Ni, Pd, Pt, Cu, Ag, Au, Zn, Cd, Hg, Pb, Ge, Ce, Sn, Al, a lanthanide, and mixtures thereof. “Substantially free of transition metal cations” means that these transition metal cations are present in such a small amount that they do not affect the properties of the ion exchanger, for example in an amount of less than 0.5 wt %, preferably less than 0.1 wt %. Minor transition metal cation impurities may not be excluded, depending on the cation exchanger used and its synthesis and storage conditions, as well as during use according to the invention. In a further preferred embodiment of the present invention, the cation exchanger is transition metal free.

In another embodiment of the present invention, the ion exchanger is an anion exchanger. It is generally conceivable to use materials in which loading with anions is possible. The anion exchanger may be an inorganic or organic anion exchanger. Preferably, the anion exchanger is an organic anion exchanger, in particular a synthetic resin ion exchanger. Organic anion exchangers are based on cross-linked polymers or copolymers containing cationically charged functional groups. According to the invention, the use of type I and type II anion exchangers is provided.

Base polymers for the anion exchangers can be, for example, cellulose, agarose, dextranes polyvinyl alcohol or polystyrene.

In one embodiment of the present invention, the at least one anion exchanger is at least one of a weakly basic anion exchanger and/or a strongly basic anion exchanger.

A “strongly basic anion exchanger” is understood to be an anion exchanger containing quaternary ammonium groups or phosphonium groups. A “quaternary ammonium group” is understood to be a functional group with a nitrogen atom which carries four organyl groups. An organyl group thereby denotes, according to the IUPAC definition, any organic substituent group, irrespective of its functionality, which bears a free valence on a carbon atom, for example CH₃—CH₂—, ClCH₂—, CH₃C(═O)—, 4-pyridylmethyl-. In a preferred embodiment, the nitrogen atom carries three groups independently selected from the group consisting of alkyl groups and aryl groups. The fourth group on the nitrogen atom represents the polymeric backbone of the anion exchanger. In another preferred embodiment, the quaternary ammonium group is a diethyl-2-hydroxyethyl-ammonium group or a sterically hindered ammonium group derived, for example, by quaternization from 1,4-diazabicyclo[2.2.2]octane (DABCO) and having high base stability. Examples of commercially available strong base anion exchangers provided in accordance with the invention include Amberlite IRA-402, Amberlite IRA-410, Amberjet 4200 CL and Dowex 1×8.

A “weakly basic anion exchanger” is understood to be an anion exchanger which contains protonable and/or protonated amino groups (—NR¹R²), wherein R¹ and R² each independently represent an organyl group. As an example, diethylaminoethyl group-bearing ion exchangers may be mentioned. Weakly basic anion exchangers are preferably used in the acidic to neutral pH range up to 100° C. For weakly basic anion exchangers, regeneration can be carried out with weak bases such as ammonia solution. Examples of commercially available weak base anion exchangers provided according to the invention are Amberlite IRA-67, Serdolit AW-1, Dowex M43, Resinex AB-1 or Lewatit MP62 and MP68.

If the anion exchanger contains weakly basic and strongly basic groups as defined above, i.e. both protonatable and/or protonated amino groups (—NR¹R²) and quaternary ammonium groups or phosphonium groups, it is referred to herein as a “medium base anion exchanger”. As an example of commercially available medium base anion exchangers provided according to the invention, Lewatit S4268 may be mentioned.

In a particularly preferred embodiment of the present invention, the anion exchanger is partially or substantially completely loaded with OH⁻ ions. By an anion exchanger partially loaded with OH⁻ ions is meant an anion exchanger in which at least 50 mol %, preferably at least 75 mol % and particularly preferably at least 80 mol % of all cationically charged functional groups have a hydroxide ion as counterion. An anion exchanger substantially completely loaded with OH⁻ ions is understood to mean an anion exchanger in which at least 90 mol %, preferably at least 95 mol % and particularly preferably at least 98 mol % of all cationically charged functional groups have a hydroxide ion as counterion.

When using anion exchangers partially or essentially completely loaded with OH⁻ ions according to the invention, their thermal stability must be taken into account. The maximum working temperature for strongly basic anion exchangers is often below 100° C. For example, for the functional group —CH₂—N(CH₃)₃⁺, the maximum working temperature is 100° C. for the Cl form and 60° C. for the OH⁻ form. For resins with the functional group —CH₂—N⁺(CH₃)₂—CH₂—CH₂—OH, the maximum working temperature of the OH⁻ form is often 35° C., but the decomposition products are not odorous. Only partial functionalization with OH⁻ often leads to products with higher thermal stability and lifetime.

The remaining functional groups carry anions as counterions. These may be selected from the group consisting of halide ions, preferably chloride, bromide and/or iodide ions, sulfate, sulfite, phosphate, hydrogen phosphate, dihydrogen phosphate, nitrate, perchlorate, fluoride, azide, borate and mixtures thereof, and are preferably selected from the group consisting of chloride, sulfate, borate (BO₃³⁻) or tetrahydroxyborate (B(OH)₄⁻) and mixtures thereof.

The anion exchanger partially or substantially completely loaded with OH⁻ ions can be obtained by washing an anion exchanger with an aqueous solution of a strong base. By a “strong base” is meant a base having a pK_{B} value of 2 or less, preferably 0 or less. The strong base may be selected from the group consisting of lithium hydroxide, sodium hydroxide, potassium hydroxide, cesium hydroxide and mixtures thereof. The aqueous solution preferably has a pH in the range of from 12 to 14, preferably from 12.5 to 14, and more preferably from 13 to 13.5.

In a further embodiment, the ion exchanger is a mixed cation and anion exchanger. By “mixed cation and anion exchanger” is meant an ion exchanger bearing both anionic and cationic groups, preferably such groups as described above.

In a further embodiment, the ion exchanger bears chelating groups. Thereby, multivalent ions in the shell of microorganisms can be selectively scavenged, e.g. with selectivity for calcium and magnesium, and thereby membrane processes of microorganisms can be disturbed. Exemplary ion exchangers having chelating groups are those whose chelating groups are derived from iminodiacetic acid, amidoximes, thiourea, aminophosphonic acids, bispicolylamine, ethylenediaminetetraacetic acid, hydroxyquinoline groups, guanidine groups, dithiocarbamate groups, hydroxamic acid groups, aminophosphoric acid groups, polyamino groups, mercapto groups, 1,3-dicarbonyl groups, thiol groups and/or cyano groups.

In another embodiment, the ion exchanger is based on naturally occurring ion-exchanging materials, such as minerals, soil or earth, or chemically modified variants thereof, or variants thereof prepared by mechanical or physical processes. Examples include soils containing clay minerals or humic acids.

In a preferred embodiment, the ion exchanger of the present invention has an ion exchange capacity of from 0.5 to 11 meq/g, more preferably from 1.5 to 6 meq/g.

A high exchange capacity in principle leads to a high uptake capacity for pathogens and the possible release of large quantities of ions potentially harmful to the pathogens, but also to high swelling in the presence of moisture and to poorer mechanical properties. The appropriate adjustment of the ion exchange capacity can be made according to the application requirements.

The ion exchange capacity indicates the number of milliequivalents of exchangeable ion per gram of dry ion exchange material and can be determined by titration. Materials with high ion exchange capacity can be loaded with high amounts of metal ions, if desired, as well as high amounts of biological contamination.

In a preferred embodiment, the ion exchanger has a high internal surface area, preferably a surface area in the range of from 15 to 1000 m²/g, preferably from 20 to 120 m²/g, measured with nitrogen and the BET method according to ISO 9277:2010. In principle, a high internal surface area enables a high ion exchange capacity.

In accordance with the present invention, the ion exchanger may comprise mixtures of the above ion exchangers.

The ion exchangers can be in the form of particles, spheres, porous molded bodies, fibres, films or papers. The molded bodies can also be functionalized with ion-exchanging groups only after manufacture. The particle size of the exchangers can vary over a wide range of sizes. For example, the median of the particle size distribution d₅₀ may be in the range of 1 μm to 10 mm, preferably in the range of 10 μm to 5 mm, more preferably in the range of 100 μm to 2 mm, and particularly preferably in the range of 300 μm to 1 mm. The median particle size distribution is the particle size at which 50% by weight of the particles are smaller than the specified particle size. The particle size distribution can be determined by a sieving method according to ASTM 0136/C136M-19 or by a sedimentation method according to ISO 13317:2001. There are no limitations regarding the width of the particle size distribution.

In a preferred embodiment, the ion exchangers are in the form of spheres having a d₅₀ in the range of 300 μm to 3 mm, preferably in the range of 300 μm to 1 mm.

The use of particles with a narrow size distribution, known in the art as “monodisperse” resins, can be advantageous from a process engineering point of view.

In another embodiment of the present invention, resins known in the prior art as gel-like ion exchangers are used. These are characterized by a microporosity with pore sizes (also often referred to as pore diameters) of up to 3 nm.

In another embodiment, resins known in the prior art as macroporous ion exchangers are used. In addition to micropores, these also contain mesopores (2-50 nm according to the IUPAC definition) and macropores (>50 nm) in the range from above 50 to 500 nm, preferably in the range from 75-250 nm. According to the invention, the pore size distributions are characterized by gas adsorption according to ISO 15901-2:2006-12.

The pore size distribution can be modified by swelling, as well as by filling the pores with solvents, salts or low molecular weight compounds. In this way, the pore sizes can be adapted to the requirements of the inventive application.

In another particularly preferred embodiment, the ion exchanger of the present invention contains water in an amount of from 10 to 200% by weight, preferably from 20 to 70% by weight, more preferably from 40 to 65% by weight, based on the total weight of the ion exchanger.

Further, the ion exchanger may be provided with hygroscopic auxiliary agents and/or hygroscopic functional groups. In particular, the ion exchanger may be provided with one or more hygroscopic auxiliary agents by the exchanger receiving the one or more hygroscopic auxiliary agents. This may be done, for example, by drying all or part of the ion exchanger and then adding the hygroscopic auxiliary agents and having all or a part thereof absorbed by the ion exchanger. Thereby, the operation of the ion exchanger can be stabilized against external influences (e.g. humidity). The hygroscopic auxiliary agents may be selected from the group consisting of hygroscopic salts such as sodium chloride, silica gel, calcium chloride, magnesium chloride and mixtures thereof or hygroscopic polymers such as superabsorbents. The hygroscopic functional groups may be selected from the group consisting of carboxylates, carboxylic acids, hydroxy groups, sulfones, sulfoxides, amino groups, and mixtures thereof.

The hygroscopic auxiliary agents may further be selected from the group of solvents having boiling points of more than 80° C. at ambient pressure, in particular solvents miscible with water. Exemplarily mentioned are glycerol, propylene glycol and propylene carbonate, in particular glycerol.

The water absorption capacity or swelling capacity can be adjusted by the degree of crosslinking. In a preferred embodiment, the ion exchanger has a degree of crosslinking in the range from 0.2 to 10%, more preferably in the range from 0.5 to 5%, most preferably in the range from 1 to 3%. The degree of crosslinking results from the molar ratio of monomers having two or more reactive groups compared to monomers having one reactive group. As used herein, a reactive group is understood to be a group that is polymerizable, for example an ethylene group. Illustrative examples of monomers having two or more reactive groups, also referred to as crosslinkers, include divinylbenzene, divinylpyridine, divinyltoluene, diallyl phthalate, ethylene glycol diacrylate, ethyleneglycol dimethacrylate, divinylxylol, divinyl ethylbenzene, divinyl sulfone, divinyl ketone, divinyl sulfide, allyl acrylate, diallyl maleate, diallyl fumarate, diallyl succinate, diallyl carbonate, diallyl malonate, diallyl oxalate, diallyl adipate, diallyl sebacate, divinyl sebacate, diallyl tartrate, diallyl silicate, triallyl tricarballylate, triallyl aconitate, triallyl citrate, triallyl phosphate, trivinylbenzene, and polyvinyl ethers of glycol, glycerol, pentaerythritol, and resorcinol, preferably divinylbenzene (DVB), trivinylbenzene (TVB), and trimethylolpropane trimethacrylate.

A variant of preferred crosslinkers are long-chain crosslinkers with molecular weights of 100 to 1000000 g/mol, which have at least two terminal polymerizable groups. These lead to the formation of ion exchangers with pore sizes >50 nm, which enable the uptake of pathogens. As a further variant, long-chain monofunctionally crosslinkable compounds, in particular compounds containing further ion-exchanging groups or being functionalizable with such groups, of molar mass 100-1000000 g/mol can be added during the crosslinking process, which form tentacle-like structures on the ion exchanger surfaces and thus improve the uptake capacity for pathogens. In the polymerization process, a preferential arrangement of such tentacles on the surface can be achieved by suitable selection of solvents and/or addition of auxiliary agents such as surfactants or hydrophilic/hydrophobic groups. Mentioned as examples be linear sulfonated polystyrene with a terminal reactive group or polymers with olefinic double bonds and sulfo groups as described in DE10022871 A1.

By varying the degree of crosslinking, the swelling properties and selectivity coefficients of ion exchange materials can be optimized for the application.

The selectivity coefficients (selectivity) are based on the equilibrium constant of the reaction: resin cation 1+cation 2=cation 1+resin cation 2. Weaker bound ions such as H⁺ or Na⁺ can be replaced by ions with higher selectivity such as Mg²⁺ or Ca²⁺, or OH⁻ by phosphate.

Typical values for selectivity coefficients range from 0.8 to 10 for cation exchangers and 0.3-175 for anion exchangers. For example, the selectivity or coefficient of selectivity of Dowex® resins for Ca²⁺ to H⁺ can be increased from 3.1 to 4.1 by increasing the crosslinker content from 4% divinylbenzene to 8% divinylbenzene. Replacement of multivalent cations in surface structures of pathogens can reduce their vitality, phosphates are also part of the surface structure of pathogens.

In general, a high degree of crosslinking leads to lower swelling of the exchangers, higher mechanical stability and lower volume change during water absorption and release, which is considered an advantage. On the other hand, lower swelling also leads to poorer accessibility of the functional groups, and a higher water content may be advantageous for the degree of deposition. In this respect, optimization is to be carried out by the person skilled in the art for the inventive application.

In addition to a wide variation of the basic structure of the ion exchangers and the type and capacity of the ion-exchanging groups, a variation of the counterions bound to the exchanger in quantity and composition is also possible.

In an exemplary embodiment of the present invention, the ion exchanger is a cation exchanger partially or substantially completely loaded with H⁺ ions, preferably a strongly acidic cation exchanger, having a water content in the range of from 10 to 75% by weight, preferably from 20 to 70% by weight, more preferably from 40 to 65% by weight, based on the total weight of the ion exchanger.

In another exemplary embodiment of the present invention, the ion exchanger is a cation exchanger partially or substantially completely loaded with H⁺ ions, preferably a strongly acidic cation exchanger, having a water content in the range of from 10 to 75 wt. %, preferably from 20 to 70 wt. %, more preferably from 40 to 65 wt. %, based on the total weight of the ion exchanger, wherein the cation exchanger is not loaded with transition metal cations selected from the group consisting of Cu, Ag and mixtures thereof and preferably is not loaded with transition metal cations selected from the group consisting of Ti, V, Cr, Mo, W, Mn, Fe, Ru, Co, Ni, Pd, Pt, Cu, Ag, Au, Zn, Cd, Hg, Pb, Ge, Ce, Sn, Al, a lanthanide and mixtures thereof or is transition metal free.

In an exemplary embodiment of the present invention, the ion exchanger is an anion exchanger partially or substantially completely loaded with OH⁻ ions, preferably a strongly acidic anion exchanger, having a water content in the range of from 10 to 75% by weight, preferably from 20 to 70% by weight, more preferably from 40 to 65% by weight, based on the total weight of the ion exchanger.

Cation exchangers and anion exchangers suitable for the present invention are also commercially available, for example under the names (manufacturers): Amberlyst® (Alfa Aesar), Dowex® (Dow Chemicals), Amberlite® (Rohm Haas), Purolite® (Purolite), Treverlite® (Chemra), Resinex® (Jacobo Carbons), Serdolit® (Serve) and Lewatit® (Lanxess).

Examples further include the commercial strongly acidic cation exchangers Amberlite IR 120, Dowex 50 WX-8, the macroporous Amberlyst 14 and Dowex 50 WX-4, the strongly basic anion exchangers Amberlite IRA-402, Amberlite IRA-410, Amberjet 4200 CL and Dowex 1×8.

Examples of commercial weakly acidic cation exchange resins are Serdolit CW-1 (Serve), Ion Exchange Resins IV (Merck) and Resinex K-H, for weak base anion exchange resins Amberlite IRA-67, Serdolit AW-1, Dowex M43, Resinex AB-1 or Lewatit MP62 or MP68, for medium base anion exchange resins Lewatit S4268. Lewatit S4268 is a macroporous and monodisperse anion exchanger.

In accordance with the present invention, the ion exchanger is preferably in solid form, for example in spherical (“bead”) or fibrous form.

The ion exchanger of the present invention may be used in conjunction with a further adsorption material, preferably selected from the group consisting of activated carbon, zeolites, silica gel and metal oxides. The further adsorption material is preferably used in an amount of not more than 25% by weight, more preferably not more than 10% by weight, relative to the ion exchanger.

In a particularly preferred embodiment of the present invention, the ion exchanger is present in a separate phase, wherein the separate phase preferably consists of the ion exchanger. This means that the ion exchanger is not present in a mixture with further adsorption materials or dispersed in a filter fleece.

The ion exchanger may also be present as a liquid or in solution, for example in water, and may be present in a hollow body, on a surface or in a pore structure. As examples, solutions of highly functionalized sulfonated polymers, such as sulfonated polyetherketones, polystyrenes or polysulfones, are mentioned. Polymers or at least oligomeric substances have the advantage over non-fixed, monomeric substances, such as benzenesulfonic acid, anisolsulfonic acids or toluenesulfonic acids, that they have no vapor pressure and are easier to handle, particularly as solids.

It may be advantageous for the use of ion exchangers according to the invention to use screen fractions of commercially available exchangers. Commercial ion exchangers are usually optimized for use in liquids. By separating fine fractions, a better flowability of packings with lower pressure drop can be achieved. Separation of coarse particles can improve the retention of pathogens by packings made of ion exchangers.

Removal and/or Reduction of Biological Contaminants

In accordance with the present invention, the ion exchanger described above is used to remove and/or reduce biological contaminants in gases and/or gas streams.

In a preferred embodiment of the present invention, the biological contaminants are selected from the group consisting of viruses, bacteria, molds, fungal spores, mites, mite droppings, pollen, and fragments of the foregoing, metabolites such as mycotoxins, proteins, RNA and DNA, preferably selected from the group consisting of enveloped viruses, non-enveloped viruses, bacteria, fungal spores and proteins and particularly preferably selected from the group consisting of coronaviruses, SARS-type viruses, SARS-CoV-2 viruses, resistant pathogens and multi-resistant pathogens. Preferably, the DNA is selected from the group consisting of plasmids and free DNA.

Examples of viruses that can be removed and/or reduced in accordance with the present invention include influenza A, adenovirus, influenza A H1 subspecies, human bocavirus, influenza A H3, human rhinovirus/enterovirus, influenza A 2009 H1N1 subspecies, coronavirus 229E, influenza B, coronavirus HKU1, respiratory syncytial virus A, Coronavirus NL63, Respiratory Syncytial Virus B, Coronavirus 0C43, Parainfluenza Virus 1, Coronavirus MERS, Parainfluenza Virus 2, SARS-CoV-2 viruses, Bordetella pertussis, Parainfluenza Virus 3, Chlamydophila pneumoniae, Parainfluenza Virus 4, Mycoplasma pneumoniae, Human Metapneumovirus and Legionella pneumophila as well as mutations of the aforementioned viruses.

Examples of bacteria that can be removed and/or reduced in accordance with the present invention include Bacillus cereus subspp., Staphylococcus epidermidis, Bacillus subtilis subspp., Staphylococcus lugdunensis, Corynebacterium spp., Streptococcus, Enterococcus, Streptococcus agalactiae, Enterococcus faecalis, Streptococcus anginosus subspp., Enterococcus faecium, Streptococcus pneumonia, Lactobacillus, Streptococcus pyogenes, Listeria, Acinetobacter baumannii, Klebsiella pneumoniae, Bacteroides fragilis, Morganella morganii, Citrobacter, Neisseria meningitides, Cronobacter sakazakii, Proteus, Enterobacter cloacae complex, Proteus mirabilis, Enterobacter spp., Pseudomonas aeruginosa, Escherichia coli, Salmonella, Fusobacterium necrophorum, Serratia, Fusobacterium nucleatum, Serratia marcescens, Haemophilus influenza, Stenotrophomonas maltophilia and Klebsiella oxytoca.

Examples of molds and fungal spores that can be removed and/or reduced in accordance with the present invention include Candida auris, Candida albicans, Candida dubliniensis, Candida famata, Candida glabrata, Candida guilliermondii, Candida kefyr, Candida lusitaniae, Candida krusei, Candida parapsilosis, Candida tropicalis, Cryptococcus gattii, Cryptococcus neoformans, Fusarium, Malassezia furfur, Rhodotorula, Trichosporon, Acremonium, Dematiaceae, Phoma Alternaria Eurotium, Rhizopus, Aspergillus, Fusarium, Scopulariopsis, Aureobasidium, Monilia, Stachybotrys, Botrytis, Mucor, Stemphylium, Chaetomium, Mycelia sterilia, Trichoderma, Cladosporium, Neurospora, Ulocladium, Paecilomyces, Wallernia, Curvularia and Penicillium.

Examples of mites that can be removed and/or reduced in accordance with the present invention include dust mites, burrowing mites, hair follicle mites, feather mites, and walking mites, as well as droppings and decay products of mites.

Examples of pollen that can be removed and/or reduced in accordance with the present invention include pollen of birch (Betula alba), alder, hazel, oak, willow, sycamore, beech, elm, maple, ash, mugwort (Artemisia) and hornbeam; grass pollen, e.g., pollen of meadow timothy (Phleum pratense), switchgrass (Poa pratense), perennial ryegrass (Lolium perenne), common bentgrass (Dactylis glomerate), wild hemp (Ambrosia artemisiifolia), common bentgrass (Anthoxanthum odoratum) and rye (Secale cereale).

In particular, the present invention is suitable for the removal and/or reduction of resistant or multi-resistant germs, since no formation of resistance is to be expected. Resistant or multi-resistant germs are understood to be bacteria and viruses which are insensitive to one or more antibiotics or antivirals.

Preferably, the gases and/or gas streams to be treated according to the present invention are air and/or air streams, preferably selected from the group consisting of room air, room air streams and breathing air streams. In a preferred embodiment, the gases and/or gas streams are breathing air streams. Preferably, the room air is selected from room air in shelters, motor vehicle interiors, air-conditioned rooms, cabins, airplanes, emergency vehicles, truck cabs, vehicles of security forces, respiratory equipment, intensive care units, staff rooms, animal husbandry rooms and other rooms in which people, animals or plants are present or located.

In a preferred embodiment of the present invention, at least 50% by weight, more preferably at least 80% by weight, especially preferably at least 95% by weight, and most preferably at least 98% by weight of the total amount of all biological contaminants are removed from the gas and/or gas stream. In another preferred embodiment of the present invention, at least 50% by weight, more preferably at least 80% by weight, particularly preferably at least 95% by weight, and most preferably at least 98% by weight of one or more of the biological contaminants selected from the group consisting of viruses, bacteria, molds, fungal spores, mites, mite droppings, antigens, pollen, and fragments of the foregoing, metabolites, such as mycotoxins, proteins, RNA, and DNA, are removed from the gas and/or gas stream. The amount of biological contaminants removed is determined by the difference in the amount of biological contaminants in the gas and/or gas stream entering the ion exchanger and in the gas and/or gas stream exiting the ion exchanger, and can be measured by the above-mentioned methods.

Preferably, the ion exchanger of the present invention is designed as a filling of a hollow body and/or as a porous molded body. Preferably, the hollow body and/or the molded body is inserted or incorporated into or connected to a respiratory mask or is incorporated or installed in or upstream of fans, air circulation systems, air conditioning systems, ventilation systems, exhaust systems and cleaning devices.

In the present invention, the removal and/or reduction of biological contaminants in gases and/or gas streams is performed by flowing a gas through a hollow body filled with the ion exchanger, for example a filter system. In this method, the gas can be passed as a gas stream through the hollow body filled with the ion exchanger, or the hollow body filled with ion exchanger resin, for example, can be actively moved through the gas.

The ion exchanger-containing filter system may comprise the following components: At least one opening for the supply of gases or gas mixtures and the same or another opening for the removal of gases or gas mixtures, wherein the ion exchanger is arranged such that the gas or gas mixture comes into contact with the ion exchanger before exiting the system.

A simple embodiment for use according to the invention is, for example, a cylinder which is filled with the ion exchanger and has an inlet and outlet at opposite ends. If necessary, the cylinder is provided with a retaining device that prevents the exchanger from being discharged from the cylinder. A retention device may be, for example, a fabric, a filtration membrane, a porous plastic member or a sintered metal.

In another embodiment, the ion exchanger is placed in a bag made of a porous material. The porous material may be a non-woven fabric, for example Tyvek™ or Gore-Tex™.

When used for breathing air, the ion exchanger can be flowed through by the pressure of the breath without any further aids, or an auxiliary force can be used to generate a pressure that results in flow. When breathing air is used, either inhaled and exhaled air may pass through the same ion exchanger in an alternating direction or inhale and exhale flow paths may be separated by a valve circuit. The ion exchanger may decontaminate both exhaled air and inhaled air. Thus, when the ion exchanger is used according to the invention in a mouth-nose protection or respiratory mask, the wearer as well as people in the vicinity are protected.

The filter system comprising the ion exchanger, for example a cylinder or other hollow body, can also be transparent for visual inspection or can be provided with measuring connectors in order to directly measure, for example, pressure, loading condition or oxygen content, or to take samples.

Indicators or dyes may also be included to indicate loading, humidity, temperature, aging or other parameters. The service life can also be determined or saved.

The filter system or hollow body may also have markings or devices to document batch, shelf life, or precise composition, or to provide instructions for use.

The cylinder or other hollow body may also be provided with quick-release closures, or screw caps, or surfaces for the application of adhesive tape or gaskets, or devices that snap into place and signal this when correctly assembled. Closures may be tamper-evident closures, closures that allow sterilization or cleaning, or porous closures that allow flushing. Separate closure systems may be included for disassembly and disposal, allowing contaminated systems to be securely closed.

Adapters may also be provided to allow regeneration or decontamination of the separation device. Additional filters for the retention of aerosols can be attached to the separator.

One embodiment may also be to fill the ion exchanger into porous bag materials and use them as a filter system. These bags or the filter system may be provided with a structure against deformation or sedimentation. The bags may be fixed in a cavity through which air or medium to be filtered flows. The fixation can be done by auxiliary materials, adhesives, welding or mechanically, e.g. by needling.

Advantageously, containers for the ion exchangers produced by means of 3D printing may also be used.

In the device for removal, the ion exchanger may also be in contact with a device that provides a mechanical bias to the ion exchanger. Such device may be, for example, a plate connected to a spring, a hydraulic or pneumatic cylinder or other volume compensating element such as a bag filled with a compressible gas. Such a bias allows the bulk density or packing density of the ion exchanger to be kept substantially constant during operation, thereby compensating for spatial changes that occur due to temperature change or moisture absorption/discharge and maintaining a filter bed without possible bypasses.

Without wishing to be bound by theory, it is assumed that the removal or reduction of biological contaminants, particularly viruses, bacteria and fungal spores, is a multi-stage process:

In a first step, the biological contaminants are adsorbed onto the surface of the ion exchanger. If the biological contamination is contained in an aerosol particle, the uptake of water from this aerosol particle can then take place. In this regard, it is advantageous if the ion exchanger contains a hygroscopic auxiliary agent or hygroscopic functional groups as described above. Subsequently, a binding of surface structures of the biological contamination to the surface of the ion exchanger takes place via one or various binding mechanisms, e.g. chemical and physical interactions, which may be reversible and/or irreversible and lead to killing of the pathogens. Examples of interactions are the formation of hydrogen bonds, ion exchange and physisorption. Depending on the porosity of the ion exchanger and the size of the pathogens, the binding can then only take place on the surface or also in the internal pore structure of the ion exchanger. Typical ion exchangers have pore sizes of 10-30 nm, so that only very small biological contaminants, e.g. non-enveloped viruses, can penetrate the structure.

Taking the spherical surface area of the ion exchanger particles or bodies as a basis (without taking into account pores and form factors), it is calculated that, for example, with an average particle size of the ion exchanger of 300 μm, one gram of ion exchanger can take up 6·10¹¹ viruses of the order of 100 nm, and with particles of 1100 μm in size, 1·10¹¹ viruses per gram. A small particle size, as described above, is therefore advantageous for achieving higher loading capacities, but leads to higher pressure losses when used for gas purification.

To increase capacity, it may also be advantageous to use macroporous ion exchangers that have a specific surface area as described above. With these or macroporous ion exchangers, pathogens can also enter the interior of the structure. By “macroporous ion exchanger” is meant an ion exchanger having pore sizes greater than 50 nm. In an exemplary embodiment, the pore size is in the range of from 50 to 500 nm, preferably from 75 to 250 nm. The use of macroporous exchangers results in higher capacity for pathogen uptake. Higher porosity can be achieved by reducing the degree of crosslinking or by increasing the molecular weight of the crosslinkers.

It is further assumed that other ions, in particular the very mobile H⁺ and OH⁻ ions, are also released from the interior of the ion exchanger and lead to or contribute to killing or inactivation of the sorbed biological contaminants. The higher the number of H⁺ or OH⁻ ions released, the more viruses can potentially be protonated and deprotonated, respectively; or the number of H⁺ or OH⁻ ions per virus present increases. Thus, a high ion exchange capacity, as described above, is advantageous. Due to the large number of ionic binding sites per bacterium or virus, it can be assumed that biological contaminants remain on the exchanger after sorption and are permanently removed from the gas stream to be purified. Elution of the biological components can be achieved by an excess of acids or bases (e.g. hydrochloric acid 5-10 wt. % or sulphuric acid 2-4 wt. % for cation exchangers or 2-5 wt. % sodium hydroxide solution for anion exchangers) or also concentrated salt solutions (NaCl 8-10 wt. %) during regeneration of the ion exchangers, which generally leads to killing of viruses and bacteria. Reversible binding by the exchange of H⁺- or OH⁻-ions or other ions present on the exchanger also leads to inactivation or reduction of pathogens. If the biological contaminants rebound from the surface due to the velocity of the gas flow, slowing of the particle velocity occurs, which facilitates sorption at other or further absorber sites. In this respect, it is advantageous to use multiple layers or packings of the ion exchangers.

The present invention is universally applicable to a wide variety of biological contaminants, particularly viruses, bacteria, fungi, and mobile genetic elements such as plasmids and free DNA. It has been found that biological contaminants containing amino acids are particularly well removed and/or reduced from gas streams.

Without wishing to by bound by theory, this mechanism is particularly universal for aerosols from sources that contain other dissolved ionic compounds, such as salts in addition to pathogens, especially from biological sources. This is the case, for example, with coughing or sneezing secretions, which typically contain a salt content (osmolarity) similar to that of other body fluids (isoosmolar to 0.9 wt % NaCl solution in the human organism), but typically at least 10% of this salt content. In the case of partial evaporation of the said secretions, higher proportions may also be present. If such aerosols now impinge on an ion exchanger according to the invention, sodium ions present in the aerosol are exchanged by the cations present on the ion exchanger. In the case of an exchanger predominantly charged with H⁺ ions, H⁺ ions are thus released. In the case of an exchanger loaded with metal cations, metal cations are released in equilibrium. In the case of an anion exchanger loaded with OH⁻ ions, chloride is bound and OH⁻ ions are released. Likewise, other bound anions such as borate ions can be exchanged in equilibrium by anion exchangers.

In the case of a cation exchanger predominantly loaded with H⁺ ions, this causes that, in the case of proteins, mixed surface charges are then completely present or, depending on the concentration present, partially present only as positive charges. Thus, carboxylate ions are converted into the free carboxylic acids, which have no charge. Exclusively or predominantly positively charged protonated nitrogen compounds remain, which bind as cations to the sulfonic acid groups of the cation exchanger. Since there are multiple binding sites, very stable binding occurs. It is assumed that this mechanism does not only apply to specific proteins but is universally applicable. DNA fragments or plasmids, which typically have phosphate groups on their surface (anions), precipitate under the resulting acidic conditions (coagulation), or can be ionically bound to the surface of protein-containing, already bound bacteria or viruses (with cationic surface). If an even stronger depletion of DNA/RNA is desired, anion exchangers functionalized e.g. with diethylaminoethanol can be used instead or additionally, e.g. in a slightly acidic environment, which directly bind phosphate groups.

By selecting the exchangers used, it is possible to remove pathogens that are sensitive to certain cations or anions or at high and low pH values.

By combining different exchangers, a broad efficacy can be achieved so that pathogens that are adapted to special living conditions can also be detected. Examples include acid-resistant bacteria, even if the route of spread is only partially via aerosols, such as Helicobacter pylori, Mycobacterium tuberculosis and enterohaemorrhagic E. coli strains (EHEC). Although these pathogens are also bound by the universal mechanism, they exhibit higher tolerance to released H⁺ ions in the case of using cation exchangers in the H⁺ form. Moreover, these pathogens are already removed from the gases or gas streams by adsorption on the ion exchanger surface.

The ion exchanger can be used in various devices or textiles.

Conceivable applications include use in garments, as a layer in mouth-nose coverings, in protective masks, in stationary and mobile air filtration devices, in combination with or in air conditioning systems, and in supply and exhaust air devices. The ion exchanger can be incorporated into exchangeable cassettes or bags that can be exchanged, collected and/or regenerated.

In a particularly preferred embodiment, the ion exchanger according to the invention is used in an air conditioning system. In this context, “air conditioning system” is understood to mean a system which conditions the air of a room by ventilation (supply of outside air or recirculating air operation), in conjunction with at least one process selected from heating, cooling, humidification and dehumidification. Thereby, it is particularly advantageous that an air conditioning system already present in a room can be used, to which the room air is fed. Thus, the integration of the ion exchanger according to the invention into an already existing system for room air conditioning is particularly simple, requires only a minimum of effort and is therefore inexpensive.

In this regard, the ion exchanger according to the invention can be integrated into the piping system of an air conditioning system in a cylinder or other hollow piece as described above, for example before the gas and/or gas stream enters the air conditioning system or after the gas and/or gas stream exits the air conditioning system. Typically, however, air conditioning systems already comprise receiving compartments or other sections for filter elements or modules. Therefore, the inventive ion exchanger can also be placed in a container or bag of suitable shape and directly integrated into the air conditioning system as a filter element or module.

In an exemplary embodiment, the ion exchanger according to the invention is used in an air conditioning system, wherein the ion exchanger is a cation exchanger partially or substantially completely loaded with H⁻ ions, preferably a strongly acidic cation exchanger, having a water content in the range of from 10 to 75% by weight, preferably from 20 to 70% by weight, more preferably from 40 to 65% by weight, based on the total weight of the ion exchanger. In this embodiment, the cation exchanger is preferably not loaded with transition metal cations selected from the group consisting of Cu, Ag and mixtures thereof, and more preferably not loaded with transition metal cations selected from the group consisting of Ti, V, Cr, Mo, W, Mn, Fe, Ru, Co, Ni, Pd, Pt, Cu, Ag, Au, Zn, Cd, Hg, Pb, Ge, Ce, Sn, Al, a lanthanide and mixtures thereof.

The packing density of the ion exchanger can also be varied. It can be realized as loose bulk, be mixed with fillers or be compressed. Compression is preferably carried out under a pressure of from 1.0 to 8 bar, preferably from 1.3 to 5 bar, particularly preferably from 1.5 to 3 bar, for example with mechanical bias. For use in compressed air systems or downstream of compressed gas accumulators, a higher compression by means of a higher initial pressure may be useful. However, for systems for room air purification, which work with pressures of e.g. 10 to 100 mbar, a lower bias is sufficient.

In a further embodiment, the air is purified by means of overpressure or underpressure, for example in a range from 1 mbar to 2 bar, preferably from 600 mbar to 1.5 bar. Typical for a use according to the invention in an air purification system is an optimization of the design with respect to filter performance, flow rate and pressure loss along the filter unit comprising the ion exchanger. Advantageously, the separation device is to be designed in such a way that pressure loss along the separation device vs. separation efficiency, duration of use vs. capacity are optimized. Typical foundations for air pollution control system design are, for example, recommendations for maximum air velocities in offices up to 0.15 m/s at 20° C. and 0.2 m/s at 26° C., which ultimately determine the size of discharge openings/cross-sections. Depending on the use of the areas and the number of users, different air exchange rates may also be necessary in order, for example, not to let the CO₂ concentration rise above 1000 ppm, max. 1400 ppm.

When using ion exchangers, the capacity design can be such that typical maintenance intervals for air handling systems, such as filter replacement, inspection of electrical systems or microbiological controls, can also be used to revise or replace the ion exchangers according to the invention. Based on a typical virus and bacteria loading of indoor air (Environ Sci Technol Lett. 2015; 2(4): 84-88) of approximately 5.7×10⁵ virus-like particles and 6.5×10⁵ bacteria-like particles per m³ of air, the particle sizes result in an approximate space requirement on the exchanger of 6.56×10⁻⁷ m² per m³ of air. For a spherical ion exchanger with a diameter of 300 μm, this results in an area of 0.006 m³/ml. With a 50% loading of the exchanger, 4575 m³ of air can be purified with one ml of ion exchanger, assuming that only a superficial monolayer loading takes place. If e.g. air purification for a room with a height of 3 m, width 10 m and length 15 m (450 m³ volume) with three air changes per hour (3*450=1350 m³/h) per day (over 8 h) (81350=10800 m³/day) on 25 days per month (10800*25=270000 m³/month), 421 g (270000 m³/month*6 months/4575 m³/g*1.19 g/ml) ion exchanger would be required.

Desirable from a technical point of view are filter efficiencies of 90% or greater, in order to achieve the greatest possible depletion during a single air exchange.

A fan shows a dependency on volume flow and pressure increase, which the skilled person gathers from the corresponding data sheets as a fan characteristic curve. For the design, the flow resistance through the system is determined; said resistance is low in a packing of ion exchangers compared to filter materials for virus retention. The high permeability results from the spherical interspaces. For monodisperse packings of ion exchangers with a diameter of 300 μm, pores in relation to a two-dimensional free area of about 23% would result, having a maximum diameter of 124 μm. Nevertheless, the separation efficiency of a packing is high, since the structure causes constant changes of direction of the air flowing through. In practice, pressure losses and flow rates are to be determined experimentally.

In still another preferred embodiment of the present invention, one or more method selected from the group consisting of filtration, humidification, drying, condensation, UV treatment, treatment with corona or plasma, treatment with high voltage, treatment with radioactive radiation, treatment by heating, treatment by cooling, ozonization, dosing of gases or liquids for treatment of the ion exchanger and/or the gas, in particular air, is carried out additionally. The treatment may be carried out permanently or at intervals. According to the present invention, the inventive method or use may be combined with one or more of the aforementioned methods to achieve even more efficient or effective decontamination. Details of these methods are described above.

“Filtration” in this context means the separation of solid or liquid substances from the gases and/or gas streams by their size. For example, the gases and/or gas streams can be passed through a filter fleece before entering the ion exchanger in order to retain larger particles (e.g. droplets or dust particles with a d₅₀≥5 μm). The filter fleece may consist of polypropylene, PTFE and/or cotton fibres.

In one embodiment, the use is in combination with humidification or drying. In order to prevent the ion exchanger from drying out, it may be advantageous to moisten air streams upstream of the ion exchanger. This can be done, for example, by dosing liquid or gaseous water, by passing through a liquid, by using a humidifier made of water-permeable material, or by using a solid impregnated with water. The humidity of the ion exchanger and the gases and/or gas streams may be monitored. The humidity of the gases and/or gas streams may be reduced by condensation. According to the present invention, the relative humidity (rH) of the gas and/or gas streams is in the range 40-100%, preferably 50-90%.

In another embodiment, the use is in combination with filters, UV light, electrical discharge, salt solutions, bactericides or virucides, liquid films with loading, heat, cold, radioactive radiation, high voltage discharge and/or plasma processes.

In one embodiment, the ion exchanger itself may be treated with UV light, electrical discharge, heat, cold, radioactive radiation, and/or high voltage discharge during operation.

The ion exchanger is preferably used in the temperature range between 0 and 100° C., since the ion exchanger preferably contains liquid water. In another embodiment, the temperature range is extended by adding solvents or salt mixtures or by carrying out the method in the absence of water.

The capacity (total amount of biological contamination that can be taken up) for the removal of biological pathogens of the ion exchanger can be monitored by time counting, overflow monitoring, pressure drop, colour indicators, sampling, detectors or based on risk assessments. It is also possible to monitor other aging parameters of the ion exchanger or of the device in which the exchanger is installed. For example, an increase in the power consumption of a fan or an increase in the pressure drop may indicate blocking of the ion exchanger, or sensors may be located in the purified air stream and/or in the supply air and measure or compare the biological load or particle quantities.

The ion exchanger may be incorporated in demountable devices that allow or facilitate recycling, regeneration or sterilisation. Alternatively, regeneration may take place in the device.

Regeneration with acid or base or other treatment processes can be used to regenerate the exchanger for the method. Preferred reactivation steps are as described above in connection with ion exchanger activation. In a preferred embodiment, one or more of drying, wetting, size separation and purging with gases or liquids is further performed.

In the apparatus, there may also be storage tanks or continuous dosing of regeneration or activation chemicals, or of mixtures, allowing operation at temperatures below 0° C. or above 100° C. Also, regeneration may be continuous or discontinuous by an applied DC or AC electrical voltage, and the ion exchanger may also be conductively contacted, for example by electrodes or by ion exchange membranes. Alternatively, a separate device may electrochemically generate the regeneration chemicals. As a side effect, an applied electric field may increase the separation efficiency of the ion exchanger.

As described above, according to the invention, air can be moved through the ion exchanger via a drive unit, the ion exchanger can be moved through the air while being fixed in a device, or a combination of both can be carried out. Air can flow through a cavity containing the ion exchanger, which is conveyed by auxiliary power or by convection, or the ion exchanger can be fixed in a fixture and the fixture can be moved through the air space. Movements and an orientation of the openings in all spatial directions are possible. It is also conceivable that the arrangement of the ion exchanger itself leads to air turbulence. Here, for example, the use as a blade of a fan or the attachment onto another rotating device can be mentioned. This may increase the amount of air that comes into contact with the exchanger, and/or reduce the amount of energy input required, and/or reduce the noise of such a device.

The effectiveness of the ion exchanger against bacteria and viruses can be verified by tests known to the skilled person in liquids, on nutrient media, by incubation or as aerosols. Therefrom, design data such as capacity and service life can be determined and the filter systems can be designed accordingly.

The method can also be adapted in terms of selection of particle size, packing density, length of packing, dwell time, temperatures, humidity by methods known to the skilled person and by routine development.

The residence time of the gases to be purified in contact with the exchanger can be adjusted over wide ranges, for example in the range from 0.01 s to 4 h, preferably in the range from 0.15 s to 1 h, particularly preferably in the range from 0.5 s to 1 min. Dwell times of fractions of a second can be realized (fast flow) or very long dwell times of minutes or hours can be realized by slow overflow, turbulence, fluidized bed arrangements or by discontinuous processes in which a volume is not continuously exchanged. The residence time distributions of the gases in the use according to the invention can be narrow or wide.

Method for the Removal and/or Reduction of Biological Contaminants

Another aspect of the present invention relates to a method for removing and/or reducing biological contaminants in gases and/or gas streams, characterized in that the gases or gas streams are passed through an ion exchanger.

The ion exchanger, biological contaminants, gases and gas streams are as described above. In the method according to the invention, the ion exchanger as described above can be used.

In a preferred embodiment of the method, the biological contaminants are selected from the group consisting of viruses, bacteria, molds, fungal spores, mites, pollen, and fragments of the foregoing, metabolites such as mycotoxins, proteins, RNA and DNA, preferably selected from the group consisting of enveloped viruses, non-enveloped viruses, bacteria, fungal spores and proteins and particularly preferably selected from the group consisting of coronaviruses, SARS-type viruses, SARS-CoV-2 viruses, resistant pathogens and multi-resistant pathogens. Preferably, the DNA is selected from the group consisting of plasmids and free DNA.

In another preferred embodiment of the method, the ion exchanger is at least one cation exchanger, optionally the cation exchanger is at least one of a weakly acidic cation exchanger and/or a strongly acidic cation exchanger. The cation exchanger may be partially or substantially completely loaded with H⁺ ions.

In still another preferred embodiment of the method, the ion exchanger is selected from the group consisting of anion exchangers, mixed anion and cation exchangers, cation exchangers loaded with transition metal ions, ion exchangers bearing chelate ligands, mixtures thereof, and mixtures thereof with cation exchangers, wherein preferably the anion exchanger is partially or substantially completely loaded with OH⁻ ions.

In another preferred embodiment of the method, the ion exchanger is at least one cation exchanger loaded with transition metal ions, wherein the transition metal ions are selected from the group consisting of cations of Ti, V, Cr, Mo, W, Mn, Fe, Ru, Co, Ni, Pd, Pt, Cu, Ag, Au, Zn, Cd, Hg, Pb, Ge, Sn, Al, a lanthanide and mixtures thereof, and preferably selected from the group consisting of cations of titanium, copper, silver and mixtures thereof. The ions may be present in various oxidation states. In particular, iron may be present as Fe⁺⁺ and/or Fe⁺⁺⁺, copper may be present as Cu⁺ and/or Cu²⁺, and titanium may be present as Ti++, Ti+++ and/or Ti++++. Particularly preferably, the cations are selected from the group consisting of Cu⁺, Cu²⁺, Ag⁺, Ti++++ and mixtures thereof.

Due to geometric effects, the pores close to the surface may be preferentially loaded with metals, while internal pores are still partially loaded with the smaller H⁺ or other ions with smaller radii.

In another preferred embodiment of the method, the ion exchanger further comprises hygroscopic auxiliary agents and/or hygroscopic functional groups.

In still another preferred embodiment of the method, the gases and/or gas streams are air or air streams, preferably selected from the group consisting of room air, room air streams and breathing air streams, preferably breathing air streams.

In a further preferred embodiment of the method, air volumes are filtered from shelters, motor vehicle interiors, air-conditioned rooms, cabins, airplanes, emergency vehicles, truck cabs, vehicles of security forces, hospitals, in particular intensive care units, staff rooms, rooms for animal husbandry or other rooms in which people, animals or plants are present or located.

In a preferred embodiment of the method, the ion exchanger is implemented as a filling of a hollow body and/or as a porous molded body. Preferably, the hollow body and/or the molded body is inserted or incorporated into or connected to a respiratory mask.

In still another preferred embodiment of the present invention, one or more methods selected from the group consisting of filtration, humidification, drying, condensation, UV treatment, treatment with corona or plasma, treatment with high voltage, treatment with radioactive radiation, treatment by heating, treatment by cooling, ozonization, dosing of gases or liquids for treatment of the ion exchanger and/or the gas, in particular air, is carried out additionally. The treatment may be carried out permanently or at intervals. According to the present invention, the method or use according to the invention may be combined with one or more of the aforementioned methods to achieve even more efficient or effective decontamination. Details of these methods are described above.

According to a further preferred embodiment of the present invention, the use of the ion exchanger according to the invention or the method according to the invention may be combined with a method for the separation or discharge of liquid condensates. The separation of condensates both further reduces pathogens and reduces or prevents condensate formation in the ion exchanger packing, which in turn could lead to higher pressure losses.

In a further embodiment of the invention, a plurality of ion exchangers are connected in parallel to provide redundancy, to enable interchangeability during operation by shutting down part of a flow, to achieve a higher retention performance, and/or to achieve distribution of air flows into a plurality of regions with only one drive unit (such as a fan).

In another preferred embodiment, multiple openings are connected in parallel for drawing in contaminated air, or it is selectively drawn in at locations of high contamination.

According to another preferred embodiment of the present invention, the use of the ion exchanger according to the invention or the method according to the invention may be carried out by connecting several ion exchangers, preferably several different ion exchangers in series. For example, a cation exchanger and an anion exchanger may be connected in series. Preferably, this allows for easier regeneration of the exchangers compared to a mixture of ion exchangers that would need to be separated prior to regeneration.

According to a further preferred embodiment of the present invention, the use of the ion exchanger according to the invention or the method according to the invention may be carried out by using mixtures of ion exchangers. This is particularly advantageous if the installation space is limited, few parts are to be used, the exchanger cannot or should not be regenerated, since the corresponding device has been operated, for example, in regulated areas such as laboratories, medical facilities or in areas with chemical, biological or radioactive contamination.

According to a preferred embodiment of the present invention, the use of the ion exchanger according to the invention or the method according to the invention can at least retain particles that can lead to a clogging of the ion exchanger in an upstream method step, in particular to prevent a clogging of the pore structure of an ion exchanger packing or ion exchanger according to the invention and thus an increase in the pressure drop across the ion exchanger packing.

In another embodiment, the ion exchangers themselves are fibers or scrims, or ion exchangers are mixed with fibers or fabrics or generally structures having pores are filled with smaller ion exchangers. In these cases, the structure itself also has a filtering effect.

According to a preferred embodiment of the present invention, the use of the ion exchanger according to the invention or the method according to the invention may include the use of a fan or compressor for air flow. Preferably, the fan or compressor can build up such a high dynamic pressure that it can force out any condensing water from the pores of the exchanger or from pores of a packing of the exchanger. Radial fans or special axial fans can be used for this purpose.

The embodiments may also differ in the spatial orientation of the ion exchange packing. While in principle an orientation in all spatial directions is possible, horizontal arrangements of the packings with flow from above or below may be preferred in order to ensure a uniform filter layer, even if different swelling of the packing occurs due to possibly less uniform exposure by water. Also conceivable as an embodiment of the invention is a packing which is flowed against from below by a filter plate and the gas flow leads to a mixing or a fluidized bed of the ion exchangers or ion exchanger particles, preferably in combination with a second filter plate which prevents discharge of the ion exchangers.

### EXAMPLES

### Example 1. Aerosol Retention Capacity

The present example illustrates the ability of the ion exchangers of the invention to retain aerosols. As a model medium for potentially virus-containing aerosols, an aerosol of a 0.9 wt. % sodium chloride solution, which is close in salt content to sneezing or coughing secretion, is used.

It is generally known that the transmission of viruses and bacteria in air currents mostly takes place in the form of aerosols, i.e. finely dispersed water droplets that can float in the air due to their size. In sizes of <5 μm, aerosols are respirable, i.e. may access the lungs. The generation of such aerosols containing human pathogens is difficult due to the high safety requirements. In biological laboratories, a variety of measures are taken to prevent the formation of aerosols. Common aerosol generators are also not designed for operation under safety cabinets or in glove boxes or too large. In this respect, a compact system was developed to demonstrate the retention of biological substances in model aerosols by ion exchangers according to the invention.

Aerosol is generated via a Pari Boy® Pro inhalation system. The system uses a compressor to generate an air flow of 3-6 l/min at 0.6-1.9 bar. With one nozzle (used: red nozzle) 0.07 to 0.18 mL of liquid per minute are nebulized. Depending on compressor flow, 74-80.6% thereof is <5 μm in size and 26-34% is <2 μm in size. The aerosol rates are determined according to ISO 27427:2013 with salbutamol.

The nebulizer of the aerosol generator is filled with 6 mL of 0.9% NaCl solution for each test. The nebulizer is operated without mouthpiece at 20° C. test temperature and a volume flow of 3 L/min (measured by water displacement from volumetric flask according to device).

The aerosol stream is fed directly into a tube with a diameter the size of the nebulizer opening (outer diameter dₐ=20 mm, inner diameter dᵢ=18 mm, length l=200 mm). The tube is configured to hold the ion exchanger. In each case 10 g of ion exchanger are filled in and fixed with a 3 mm thick layer of cotton wadding. As an alternative to cotton wadding, 20 mm filter foam with 30 ppi (pores per inch, which corresponds to 30 pores per 25.4 mm) made of polyurethane can be used (filter tube).

At least 2, typically 3 to 4 collection bottles are arranged downstream of this tube. These are each 100, 250, 500 or 1000 mL capacity, top-sealed polypropylene wide-neck bottles, which are provided with two connections made of stainless steel tube (outer diameter dₐ=6 mm) at the sides. The inlet connection protrudes to the bottom of the bottle and is slit at the bottom 4 times 5 mm upwards to create turbulence. The outlet connection is short and is used for gas discharge. The advantage of this arrangement is that it is compact and has no sharp-edged parts. In addition, it can be assembled and disassembled without tools. All parts can be disinfected in a bath and are autoclavable.

To carry out the experiment, the first collecting bottle (250 ml) after the sample tube is filled with 150 mL of demineralised water in which a conductivity electrode is completely immersed. Two more empty bottles are placed after the collection bottle to collect liquid droplets carried along by the air flow. During the experiment, the air flow is measured downstream of the system and, after the experiment, the liquid residues are removed from the evaporator and weighed back.

In the course of the experiment, the conductivity in the first collection bottle is recorded against time for an experimental period of 30 minutes. The conductivity follows the NaCl concentration in a linear fashion and is 64 mS·L·mol⁻¹ at 25° C. The 0.9 wt % NaCl solution used has a conductivity of 13.66 mS cm⁻¹ at room temperature (20±3° C.). The increase in conductivity is determined by linear extrapolation. The retention in % is calculated as (1−(slope sample/slope empty tube))*100.

The following ion exchangers are used:

(1) Purolite® MB 400, a polystyrene-based mixed bed resin composed of a strongly basic type I anion exchanger with quaternary ammonium ions in the hydroxide form and a strongly acidic gel cation exchanger with sulfonic acid groups in the hydrogen form. The capacity of the exchanger is about 1.9 eq/L. They are spherical balls of a gel of 300 to 1200 μm in size, a water content of 65% and a bulk density of 705-740 g/L.

(2) Amberlite® IRC 120, a strongly acidic cation exchanger in the H⁺ form. It is a gel ion exchanger based on styrene-divinylbenzene with a water content of 50%. The exchanger capacity is 1.8 eq/L. The bulk density is 785 g/L. The particle size of the exchanger is 95% between 300 and 1200 μm.

A Hygostar type IIR/PP>98% BFE medical mask is used as a reference (positive control). The test section corresponds to the inner diameter of the tube.

As a negative control (empty tube), the tube is filled with cotton wool.

Results are reported relative to retention in the negative control and are summarized in Table 1.

TABLE 1
Retention of aerosols

| | NaCl permeation/μS | Relative |
|---|---|---|
| Sample name | cm⁻¹ min⁻¹ | retention/% |
| Negative control (cotton wool) | 8.764 | 0 |
| (1) Purolite MB 400 | 0.485 | 94.5 |
| (2) Amberlite IRC 120 | 0.194 | 97.8 |
| Positive control (Hygostar) | 0.016 | 99.8 |

All backweighing results show that more than 4 g of NaCl solution was atomized during the test duration. It could thus be shown that the ion exchangers according to the invention can retain more than 90% of airborne aerosols from a volume flow.

### Example 2. Preparation of H⁺ and Metal Ion Loaded Ion Exchangers

For the following tests, the strongly acidic cation exchange resin Amberlite® IRC 120 Na with a sodium content of 9.2% based on the dry mass of the exchanger is used. This is a gel based on sulfonated polystyrene in the Na form, crosslinked with divinylbenzene. The exchange capacity is >2 eq/L, the water content is 49% and the density is 840 g/L. The resin contains <2% of particles <300 μm and <4% of particles >1180 μm.

To prepare the H⁺ form, the cation exchange resin is packed into a column. Glass columns with an inner diameter of 20 mm and a length of 200 mm (up to 20 g filling) and a column with an inner diameter of 40 mm and a length of 1000 mm (for Cu(1)) are used. The columns have filter plates with porosity 0 (ISO P 250, nominal width of pores 160-250 μm). It is rinsed with hydrochloric acid of pH 0. 100 mL of 1N hydrochloric acid (0.1 mol) to 10 g resin (0.024 mol) is used (excess) to ensure as complete a conversion as possible to the H⁺ form. The exchanger is then rinsed with deionized water until the eluate is no longer acidic and has a conductivity <20 μScm⁻¹. The loaded resin is removed and designated as H-(1). It has a water content of 51%.

To prepare a metal ion-loaded ion exchanger, the ion exchange resin in the H⁺ form thus obtained is packed into a column and rinsed with a solution of an appropriate metal salt. The flush rate is one bed volume per hour. Flush rates up to 50 bed volumes per hour are possible, but result in lower loadings. The exchanger is then rinsed with deionized water until the eluate has a conductivity <20 μScm⁻¹. The loaded resin is removed.

In the Ti-(1) variant, the initially turbid solution is left to stand overnight on the ion exchanger. As a result of the release of H⁺ ions, the turbidity disappears, as a sulfuric acid solution is formed. The calculation of the degree of loading by the respective metal is based on an ion exchange capacity IEC (Ion Exchange Capacity) of 4 meq/g for Na⁺ with respect to the dry mass of the ion exchanger (2 meq/g with respect to moist ion exchanger). This is a comparative observation, since the exchanger capacity refers to Na⁺ ions and was not determined for the respective ions. It can be assumed that not all exchanger sites accessible for sodium are accessible for the larger metal ions and that loading of the geometrically inner sites takes place kinetically delayed.

The resins obtained and the corresponding loadings are listed in Table 2.

TABLE 2
Cation exchange resins obtained from Amberlite ® IRC 120 Na.

| | | | | Ion/metal content |
|---|---|---|---|---|
| | | | | in [wt.-%]¹ |
| | Educt H-(1) | Metal salt | Metal salt | (loading |
| Sample | [g]¹ | used | [g/ml water] | degree metal) |
| Zn-(1) | 11.7 | Zinc(II) acetate | 10.55/100 | Zn^{II}/19.5 |
| | | dihydrate | | (75 mol %) |
| Cu-(1) | 340.3 | Copper(II) | 350.0/2000 | Cu^{II}/18.2 |
| | | sulfate | | (72 mol %) |
| | | hexahydrate | | |
| Ag-(1) | 9.9 | Silver(I) | 8.04/100 | Ag^{I}/34.1 |
| | | nitrate | | (79 mol %) |
| Fe-(1) | 11.2 | ferric chloride | 13/100 | Fe^{III}/21.5 |
| | | hexahydrate | | (96 mol %) |
| Ti-(1) | 20 | Ti(IV)O | 7.61/150 | Ti^{IV}/7.5 |
| | | sulfate | | (39 mol %) |
| Sn-(1) | 20 | Tin(II) | 10.27/100 | Sn^{II}/25.4 |
| | | sulfate | | (53 mol %) |

¹based on the dry mass of the ion exchanger.

The percentage loading is highest at 96 mol-% for Fe³⁺. The respective percentage difference is present in the H⁺ form (e.g. 4 mol-% for Fe³⁺).

### Example 3: Antiviral Activity of Ion Exchangers

In addition to aerosol retention, it is central to the operation of the invention that retained pathogens are sorbed and inactivated at the ion exchangers.

The antiviral activity of the metal-loaded ion exchangers as well as the acidic ion exchanger H-(1) can be investigated in suspension experiments, for example as described as follows: Human coronavirus HCoV-OC43Rluc is used as the virus. Virus concentrate is incubated for 30 min with the ion exchangers prepared in Example 2 (100 mg/mL) at room temperature in a shaker. The polymer is then removed by centrifugation, the supernatant is removed and titrated onto 293 T cells. Cells are lysed 30 h after inoculation and Renilla assays are performed on the lysates. The reduction of viral activity is calculated from the Renilla signal of the reporters in RLU (relative light units) compared to the reference at a dilution of the supernatant of 1:10. It can be shown that the cation exchanger H-(1) in the H⁺ form as well as Ti-(1) and Sn-(1) are most effective. The very effective form Ti-(1) is only partially converted and is about 60% in the H⁺ form. Cu-(1) as well as Ag-(1) cation exchangers can also be very effective.

### Example 4: Antibacterial Activity of Ion Exchangers

The antibacterial activity of the ion exchangers can be studied in suspension experiments, for example as described as follows: The following bacterial strains are used: Streptococcus pneumoniae, Haemophilus influenzae and Staphylococcus aureus, methicillin resistant. Mix each 100 mg of ion exchanger with 1 mL of a PBS buffer solution and one bacterial colony taken directly from an agar plate. Then incubate for 2, 10 and 30 min at 37° C. on the shaker. Cu-(1) is used as ion exchanger. S. aureus, methicillin resistant (Gram positive) can be successfully killed after 2 min incubation, no growth is seen after 72 h. For Streptococcus pneumoniae and Haemophilus influenzae, delayed growth can be observed after 10 and 30 min of incubation. In the light microscope, bacteria can be found on the ion exchanger surface.

### Example 5: Determination of Characteristic Curves for the Design of Ventilation Systems

To determine ventilation characteristics, packings of ion exchangers are installed in pipes and the pressure drop is measured as a function of the flow velocity. H-(1) is used as an example of a strongly acidic cation exchanger. The water content is 50%, 95% of the particles are between 0.3 and 1.2 mm in size. An adjustable duct fan is used with a fan speed of up to 3800 rpm and a free-flowing air stream (manufacturers specification) of up to 561 m³/h to 565 Pa.

The suction takes place free-flowing via a pipe (diameter 125 mm, length 50 cm). The ion exchanger packing is installed on the pressure side in a pipe with an inner diameter of 11.25 cm. The packing is held between two discs of open-pored filter foam (polyester-based polyurethane foam) with 20 mm thickness and with a pore size of 30 ppi, as they are used as pre-filters in air-conditioning systems.

The pressure difference along the packing and the volume flows are determined with a differential pressure gauge and pitot tube anemometer Trotec TA 400. For this purpose, measuring connections are installed in the flow pipe upstream and downstream of the ion exchanger packing. For linearization, the flow after the packing is guided through a constricted cylindrical pipe with a diameter of 64 mm.

The pressure drop is determined as a function of the flow velocity at 20.7° C. with air. With the fan at maximum power, air velocities of maximum 13.5 m/s result during free blowing in the device, which corresponds to 483 m³/h.

TABLE 3
Pressure drop vs. flow velocity for prefilter made of 2 layers
of filter foam, each with 2 cm thickness and 30 ppi

| | Velocity [m/s] | |
|---|---|---|
| Pressure drop/Pa | in 11.25 cm (d) tube | Volume flow m³/h |
| 281 | 4.23 | 151 |
| 226 | 3.9 | 140 |
| 155 | 3.25 | 116 |
| 65 | 2.34 | 84 |
| 28 | 0.93 | 33 |

TABLE 4
Pressure drop vs. flow velocity for packing thickness
of 1 cm ion exchanger H-(1) (100 g) between two layers
of filter foam, each with 2 cm thickness and 30 ppi

| | Velocity [m/s] | |
|---|---|---|
| Pressure drop/Pa | in 6.4 cm (d) tube | Volume flow m³/h |
| 336 | 5.61 | 65 |
| 209 | 4.72 | 55 |
| 92 | 2.36 | 27 |
| 41 | 0.2 | 2 |

These data show that packings of commercially available ion exchange resins can be used in conventional ventilation systems with moderate pressure losses at high volume flow rates.

### Example 6: Antiviral Efficacy Against SARS-CoV-2 Viruses

The studies are performed with Human 2019-nCoV strain 2019-nCoV/Italy-INMI1, clade V, sequence see GenBank (SARS-CoV-2/INM11 isolates/2020/Italy: MT066156).

The antiviral efficacy of H-(1) is studied by the following method: Virus (in DMEM, Dulbecco's Modified Eagle's Medium, high glucose, Thermo Fisher 41965) with a titer of (>10⁶ TCID₅₀/ml) is used (TCID=tissue culture infectious dose, the dose necessary to induce infection in 50% of the cell cultures). The virus suspension is incubated with ion exchanger H-(1) (10 wt %) under shaking. The virus titer of SARS-CoV-2 is measured in the upper phase after separation from ion exchanger H-(1). Samples are taken 2, 10 and 30 minutes after addition of the ion exchanger. A 10-fold dilution series (10⁻¹ to 10⁻⁶) of supernatants is used to infect a Vero E6 cell monolayer in a 96-well plate. Cells are cultured for 72 hours and infection is quantified microscopically by cytopathic effect (Leica microscope). The virus titer is determined by the Reed-Muench method. A virus suspension without the addition of an ion exchanger is used as a control. The results are given in Tables 5 to 11.

Bound viruses of the 30 min incubated sample are eluted from H-(1) with 0.9 wt % NaCl solution. The eluate is diluted (minimum 1:10 final in complete culture medium) and tested for replication ability in Vero E6 cells. NaCl 0.9 wt % and the virus in NaCl 0.9 wt % are tested at the same dilution as a control.

The virus titer is determined in Vero E6 cells (Cercopithecus aethiops, kidney, ATCC CRL-1586). The cell line is routinely maintained in DNEM culture medium with the addition of 1% glutamine, 1% penicillin/streptomycin and 10% FBS (fetal bovine serum).

Viral replication capacity is determined as follows: Exponentially growing Vero E6 cells are seeded into a 96-well plate at optimal density in complete medium. 24 h later, cells are exposed to eluates of ion exchanger H-(1) and controls. Another control is cells infected with SARS-CoV-2 (multiple of infection, 0.01 TCID₅₀/cell). The cells are then cultured for 72 h. Two replicates for each concentration are examined. At the end of the incubation period, the antiviral activity is quantified on the one hand by an ELISA assay (Enzyme-linked Immunosorbent Assay (ELISA), an antibody-based detection method (assay)) (Sino Biological, quantifying SARS-CoV-2 nucleoprotein) and on the other hand by microscopic control of cytopathic effect (Images: Leica Microscope). The results are shown in Table 12.

The following tables 5 to 10 show the readout of the experiment to determine the virus titer after 72 hours. Incubation with Vero E6 cells. Infected (+) and uninfected (−) wells are indicated for the 6 replicates of 10-fold serial dilutions tested for each supernatant collected. The infection status of each well was assessed by observing the cytopathic effect on the microscope. Score: infected “+”, not infected “−”, First row each dilution of supernatant.

TABLE 5
2-minute incubation time. Control suspension
virus (not treated with H-(1))

| 10⁻¹ | 10⁻² | 10⁻³ | 10⁻⁴ | 10⁻⁵ | 10⁻⁶ | 10⁻⁷ | 10⁻⁸ | 10⁻⁹ |
|---|---|---|---|---|---|---|---|---|
| + | + | + | + | + | − | − | − | − |
| + | + | + | + | − | − | − | − | − |
| + | + | + | + | − | − | − | − | − |
| + | + | + | + | − | − | − | − | − |
| + | + | + | + | − | − | − | − | − |
| + | + | + | + | − | − | − | − | − |

TABLE 6
2-minute incubation time. Virus suspension
treated with 10 wt % H-(1)

| 10⁻¹ | 10⁻² | 10⁻³ | 10⁻⁴ | 10⁻⁵ | 10⁻⁶ | 10⁻⁷ | 10⁻⁸ | 10⁻⁹ |
|---|---|---|---|---|---|---|---|---|
| + | + | − | − | − | − | − | − | − |
| + | + | − | − | − | − | − | − | − |
| + | + | − | − | − | − | − | − | − |
| + | + | − | − | − | − | − | − | − |
| + | + | − | − | − | − | − | − | − |
| + | + | − | − | − | − | − | − | − |

TABLE 7
10-minute incubation time. Control suspension
virus (not treated with H-(1))

| 10⁻¹ | 10⁻² | 10⁻³ | 10⁻⁴ | 10⁻⁵ | 10⁻⁶ | 10⁻⁷ | 10⁻⁸ | 10⁻⁹ |
|---|---|---|---|---|---|---|---|---|
| + | + | + | + | − | − | − | − | − |
| + | + | + | + | − | − | − | − | − |
| + | + | + | + | − | − | − | − | − |
| + | + | + | + | − | − | − | − | − |
| + | + | + | + | + | − | − | − | − |
| + | + | + | + | − | − | − | − | − |

TABLE 8
10-minute incubation time. Virus suspension
treated with 10 wt % H-(1)

| 10⁻¹ | 10⁻² | 10⁻³ | 10⁻⁴ | 10⁻⁵ | 10⁻⁶ | 10⁻⁷ | 10⁻⁸ | 10⁻⁹ |
|---|---|---|---|---|---|---|---|---|
| − | − | − | − | − | − | − | − | − |
| + | − | − | − | − | − | − | − | − |
| − | + | − | − | − | − | − | − | − |
| − | − | − | − | − | − | − | − | − |
| − | − | − | − | − | − | − | − | − |
| − | − | − | − | − | − | − | − | − |

TABLE 9
30-minute incubation time. Control suspension
virus (not treated with H-(1))

| 10⁻¹ | 10⁻² | 10⁻³ | 10⁻⁴ | 10⁻⁵ | 10⁻⁶ | 10⁻⁷ | 10⁻⁸ | 10⁻⁹ |
|---|---|---|---|---|---|---|---|---|
| + | + | + | + | + | − | − | − | − |
| + | + | + | + | + | − | − | − | − |
| + | + | + | + | − | − | − | − | − |
| + | + | + | + | − | + | − | − | − |
| + | + | + | + | + | − | − | − | − |
| + | + | + | + | + | − | − | − | − |

TABLE 10
30-minute incubation time. Virus suspension
treated with 10 wt % H-(1)

| 10⁻¹ | 10⁻² | 10⁻³ | 10⁻⁴ | 10⁻⁵ | 10⁻⁶ | 10⁻⁷ | 10⁻⁸ | 10⁻⁹ |
|---|---|---|---|---|---|---|---|---|
| − | − | − | − | − | − | − | − | − |
| − | − | − | − | − | − | − | − | − |
| − | − | − | − | − | − | − | − | − |
| − | − | − | − | − | − | − | − | − |
| − | − | − | − | − | − | − | − | − |
| − | − | − | − | − | − | − | − | − |

TABLE 11
Virus titer calculated by Reed-Muench method for the
different test conditions for samples incubated with
10 wt % H-(1) and control samples without treatment.

| | | Log reduction compared to |
|---|---|---|
| Test condition | Virus titer | control |
| 2 min incubation, control | 3.98E+05 | 2.1 |
| 2 min incubation, H-(1) | 3.16E+03 | |
| 10 min incubation, control | 3.98E+05 | >3.6 |
| 10 min incubation, H-(1) | <1.00E+02 | |
| 30 min incubation, control | 2.29E+06 | >4.36 |
| 30 min incubation, H-(1) | <1.00E+02 | |

The following Table 12 shows antiviral activity data of test samples from the virus replication capacity determination experiment. Mean and standard deviation values of SARS-CoV-2 nucleoprotein are shown for each test condition.

TABLE 12
Antiviral activity (SARS-CoV-2 nucleoprotein, ELISA). M.o.i.
(multiplicity of infection) means the number of TCID₅₀ (infectious
dose of tissue cells 50%, i.e. the amount of virus infecting
half of the culture cells)/cells used for infection.

| | SARS-CoV-2 nucleoprotein in ng/mL | |
|---|---|---|
| Test condition | Mean value | Standard deviation |
| Control (0.01 m.o.i) | 1125 | 202 |
| Virus + NaCl 0.9% | >50 | — |
| NaCl 0.9% (negative control) | <0.07 | — |
| Eluted fraction of H-(1) | 4.07 | 0.29 |
| 30 min Virus incubation | | |

The eluted fraction comprises hardly any detectable viral nucleoprotein, indicating almost complete inactivation of the virus.

### Example 7. Impactor Test Room Air with Microscopic Evaluation

Filter tubes according to Example 1 are tested in impactor tests for the retention of particles from room air. A particle collector PS 30 from Holbach Umweltanalytik and adhesively coated slides are used for this purpose. Sampling is carried out by means of a diaphragm pump, which is regulated to a volume flow of 5 litres per minute by means of a dosing valve. A sample is taken for 30 min, which corresponds to a total volume of 150 L. The samples are examined using a light microscope (Leica, at magnification 200-1000) and particles are identified as far as possible. Comparatively, the following results were obtained (Table 13):

TABLE 13
Results of impactor test.

| sampling location | Without ion exchanger | With ion exchanger H-(1) |
|---|---|---|
| Outdoor Air, | Grass pollen, various | Sporadically, |
| Meadow, May | Rye Pollen | not identifiable |
| | Fungal spores | |
| | Fiber residues | |
| Indoor air, | Fiber residues | Sporadically, |
| bedroom | Mites | not identifiable |
| | Sporadically spores | |
| | Dust, miscellaneous | |

The results show that packings of ion exchange resins also retain various particles with sizes of several μm and pollen.

### Example 8. Retention of DNA from Aerosol by Ion Exchanger

0.1 g deoxyribonucleic acid, low molecular weight, from salmon sperm (CAS number 1000403-24-5, Sigma Aldrich 31149) sheared to ≤2000 bp (base pairs) is suspended in 5 ml distilled water in a tube shaker at room temperature for 10 min.

The suspension is nebulized as an aerosol in a device as described in example 1 immediately after shaking. After the filter tube, 2 bottles each containing 100 ml of distilled water are arranged to collect the DNA. The amount of DNA transferred is determined gravimetrically as dry residue. Determined is the retention compared to the blank tube in %, calculated as (dry residue sample-zero sample)/(dry residue blank tube+filter foam 2×2 cm 30 psi zero sample). 10 g of ion exchanger is used in each case.

Preparation of anion exchanger on glass beads (A-glass-(1)): 1.0518 g PVA (Polinol 1000) is dissolved at 60° C. in 50 ml water. After cooling, 1.3 g of 37% HCl is added. 0.7218 g diethylaminoacetaldehyde dimethyl acetal is added and kept at 60° C. for 1 h. Then 0.5315 g of butyraldehyde dimethyl acetal is added under stirring. After about 5 min, a white, spherical polymer precipitates. This is washed with water until the supernatant no longer reacts acidically, and dried. 1.165 g of dried product is obtained. The product is suspended or dissolved in 20 ml ethanol at 40° C. and mixed with 10 g glass spheres (3M® Glass Bubbles K 1.65 μm). Ethanol is evaporated under stirring and the resulting mass is ground in a mortar.

Preparation of anion exchanger with diethylaminoethanol function (DEAE-1): An ion exchange resin is prepared according to JPS5811046 A, but in deviation from the protocol, diethylaminoethanol is used instead of morpholine.

DEAE-Sepharose® CL-4B (DEAE-2) with the functional group —OCH₂CH₂N⁺H(CH₂CH₃)₂ and chloride as counterion with an ion exchange capacity of 0.13-0.17 meq/mL is flushed from a suspension of 20% ethanol with 0.5 molar sodium chloride solution into the empty tube with filter foam and the liquid is allowed to drain. DEAE-2 is composed of particles from 45 to 165 μm in size.

Purolite® MB 400 is used as a mixture of cation and anion exchangers, consisting of a strongly basic type I anion exchanger with quaternary ammonium ions in the hydroxide form and a strongly acidic gel cation exchanger with sulfonic acid groups in the hydrogen form (abbreviated: HOH-(1)).

As a negative control, 200 ml of demineralised water as used is dried. The dry residue of 0.5 g obtained corresponds to the detection limit of the method. A pipe with 2×2 cm filter foam 30 ppi (pores per inch, which corresponds to 30 pores per 25.4 mm) made of polyurethane is used as the blank pipe. The results are summarized in Table 14.

TABLE 14
Results DNA retention.

| Sample name | Dry residue/mg | Relative retention/% |
|---|---|---|
| Empty pipe with filter foam | 45 | 0% |
| H-(1) | 0.8 | 99 |
| A-glass-(1) | 1.5 | 98 |
| HOH-(1) | 1.2 | 98 |
| DEAE-(1) | 0.8 | 99 |
| DEAE-(2) | 0.7 | 100 |

The ion exchangers all show a very high retention for DNA in aerosol form.

### Example 9: Retention of Aerosols with Glycerinated Ion Exchanger

20 g of an ion exchanger H-(1) are mixed with 5 g of glycerol (>99% purity) and dried at 110° C. until constant weight. The obtained ion exchanger H-(2) is filled into a filter tube as described in Example 1 and closed on both sides with filter foam of a thickness of 2 cm, porosity 30 ppi (pores per inch, corresponding to 30 pores per 25.4 mm). An aerosol stream is added by means of a 5 wt.-% NaCl solution at 20° C. and a volume flow of 1.5 cm/s (corresponding to 230 ml/min). The aerosol is generated with a Palas® PLG1000 aerosol generator, and the penetrating aerosols are detected fractionally by size with a Palas® Promo 1000 aerosol spectrometer. Depending on the aerosol size, the following retention rates result (Table 15):

TABLE 15
Results NaCl aerosol retention with H-(2).

| Aerosol size/nm | Relative retention/% |
|---|---|
| 100 | 80 |
| 150 | 90 |
| 1000 | 99 |

Retention increases with increasing size of aerosol particles.

### Example 10 Further Possible Uses

6.1) The cation exchange resin H-(1) is filled into a cylindrical piece of tubing, which is closed on both sides with a filter fabric. The tube is connected to a conventional reusable breathing mask by means of a flexible corrugated tube.

6.2) A cation exchange resin according to example 6.1 is sealed in a bag made of a porous material, such as Tyvek™ or Gore-Tex™ of dimension 10*10 cm. A filter fleece is inserted into the bag to spatially fix the resin. The bag is inserted between two layers of a filtering mouth protection. A special bag may be provided for insertion so that separation/cleaning can be performed. Alternatively, the bag may be fixed in its position by a holding device, adhesive tape, sewing or gluing.

6.3) A device such as examples 6.1 and 6.2, in admixture with ion exchange resins, using ions of the following elements or mixtures thereof: Ti, V, Cr, Mo, W, Mn, Fe, Ru, Co, Ni, Pd, Pt, Cu, Ag, Au, Zn, Cd, Hg, Pb, Ge, Ce, Sn, Al, or a lanthanide.

6.4) A device according to examples 6.1-6.3, in which a mixed ion exchanger is used and borates are used as anion.

6.5) A device according to examples 6.1-6.4, in which a mixed ion exchanger is used and NH₄⁺ is also used as cation.

6.6) Use of a device according to example 6.1-6.5 for the reduction of bacteria and viruses from a respiratory air stream. The retention of bacteria and viruses is assessed according to EN 14126, analogous to ISO 16603 and ISO 22610.

6.7) Use of a device according to examples 6.1-6.5 for the retention of corona viruses.

6.8) Use of a device according to examples 6.1-6.5 for the retention of SARS-CoV 2.

6.9) Use of a device according to example 6.1-6.5 to reduce the bacteria and virus count in distilled water by flowing the water through the device.

6.10) Use of a device according to example 6.1-6.8 as a filtering device upstream of air volumes such as shelters, car interiors, air conditioning systems, cabins, airplanes, emergency vehicles, truck cabs, vehicles of security forces, respiratory equipment, intensive care units, staff rooms, rooms for animal husbandry or other rooms in which people, animals or plants are present or located.

6.11) Protection of air or liquid carrying devices against germination or biological contamination by devices according to example 6.1-6.8.

6.12) Ventilation valve according to example 6.1-6.8.

6.13) An anion exchanger is used which is obtained by functionalizing a scaffold polymer containing halogen end groups with diethylaminoethanol. The anion exchanger may be used partially or substantially completely in the hydroxide form.

6.14) Advantageous are also anion exchangers functionalized with sterically hindered amines, so that an increased base stability of the exchangers results and the exchangers also remain thermally stable in OH form or can be sterilized. An example of such amine is DABCO, diazabicyclooctane.

15) A ceiling fan is equipped with blades filled with ion exchange resin.

16) A flow-through molded body filled with ion exchange resin is arranged on a rotating device and air flow is generated due to rotation. This device may be combined with a humidifying device.

Described below are further embodiments of the present invention.

According to embodiment 1, the present invention relates to the use of one or more ion exchangers for the reduction and/or removal of biological contaminants in gases and/or gas streams.

According to embodiment 2, the use according to embodiment 1 is characterized in that the biological contaminants are selected from the group consisting of viruses, bacteria, molds, fungal spores, mites, mite residues, mite droppings, pollen as well as fragments of the foregoing, metabolic products, such as mycotoxins, proteins, RNA and DNA, are preferably selected from the group consisting of enveloped viruses, non-enveloped viruses, bacteria, fungal spores and proteins and particularly preferably selected from the group consisting of coronaviruses, SARS-type viruses, SARS-CoV-2 viruses, resistant pathogens and multiresistant pathogens. Particularly preferred is the use of one or more ion exchangers for the reduction and/or removal of coronaviruses in gases and/or gas streams.

According to a further embodiment, the use according to any one of the preceding embodiments is characterized in that the ion exchanger comprises at least one cation exchanger, optionally the cation exchanger being at least one of a weakly acidic cation exchanger, a strongly acidic cation exchanger, or a mixture thereof.

According to a further embodiment, the use according to any one of the preceding embodiments is characterized in that the strongly acidic cation exchanger is an organic, particularly preferably a synthetic resin ion exchanger, having sulfonic acid and/or sulfonate groups, preferably selected from a cross-linked polystyrene sulfonate or cross-linked poly(2-acrylamido-2-methyl propanesulfonic acid) (polyAMPS).

According to a further embodiment, the use according to any one of the preceding embodiments is characterized in that the cation exchanger is partially or substantially completely loaded with H⁺ ions.

According to a further embodiment, the use according to any one of the preceding embodiments is characterized in that the ion exchanger is selected from the group consisting of anion exchangers, mixed anion and cation exchangers, cation exchangers loaded with transition metal ions, ion exchangers bearing chelate ligands, mixtures thereof and mixtures thereof with cation exchangers, wherein preferably the anion exchanger is partially or substantially completely loaded with OH⁻ ions.

According to a further embodiment, the use according to any one of the preceding embodiments is characterized in that the ion exchanger is at least one cation exchanger loaded with transition metal ions, wherein the transition metal ions are selected from the group consisting of cations of Ti, V, Cr, Mo, W, Mn, Fe, Ru, Co, Ni, Pd, Pt, Cu, Ag, Au, Zn, Cd, Hg, Pb, Ge, Sn, Al, or a lanthanide, and mixtures thereof, and preferably selected from the group consisting of cations of copper, silver, titanium, and mixtures thereof.

According to a further embodiment, the use according to any one of the preceding embodiments is characterized in that the ion exchanger is present in solid form, preferably as an organic ion exchanger and particularly preferably as a synthetic resin ion exchanger, wherein the ion exchanger may be present in particulate form, as a flow-through fill and/or as an insert in a mouth-nose protection. The ion exchanger in solid form is preferably insoluble in water.

According to a further embodiment, the use according to any one of the preceding embodiments is characterized in that the ion exchanger further comprises hygroscopic auxiliary agents and/or hygroscopic functional groups, or is used in combinations with such auxiliary agents.

According to a further embodiment, the use according to any one of the preceding embodiments is characterized in that the gases and/or gas streams are air and/or air streams, preferably selected from the group consisting of room air, room air streams and breathing air streams, wherein the room air and room air streams are preferably selected from room air and room air streams in shelters, motor vehicle interiors, air-conditioned rooms, cabins, airplanes, emergency vehicles, truck cabs, vehicles of security forces, respiratory equipment, intensive care units, staff rooms, rooms for animal husbandry and other rooms in which humans, animals or plants are present or located.

According to a further embodiment, the use according to any one of the preceding embodiments is characterized in that the ion exchanger is designed as a filling of a hollow body and/or as a porous molded body, the hollow body and/or the molded body preferably being inserted or incorporated into or being connected to a respiratory mask.

According to a further embodiment, the use according to any one of the preceding embodiments is in combination with one or more of the methods selected from the group consisting of filtration, humidification, drying, condensation, UV treatment, corona or plasma treatment, high voltage treatment, radioactive radiation treatment, treatment by heating and treatment by cooling.

According to a further embodiment or aspect, the present invention relates to a method for removing and/or reducing biological contaminants in gases and gas streams by means of one or more ion exchangers, characterized in that the gases or gas streams are brought into contact with an ion exchanger.

According to a further embodiment, the method according to any one of the preceding embodiments is characterized in that the biological contaminants are selected from the group consisting of viruses, bacteria, molds, fungal spores, mites, mite residues, mite droppings, pollen and fragments of the foregoing, metabolic products, such as mycotoxins, proteins, RNA and DNA, are preferably selected from the group consisting of enveloped viruses, non-enveloped viruses, bacteria, fungal spores and proteins and particularly preferably selected from the group consisting of coronaviruses, SARS-type viruses, SARS-CoV-2 viruses, resistant pathogens and multi-resistant pathogens. Particularly preferably, the method is employed using one or more ion exchangers for the reduction and/or removal of coronaviruses in gases and/or gas streams.

According to a further embodiment, the method according to any one of the preceding embodiments is characterized in that the ion exchanger comprises at least one cation exchanger, optionally the cation exchanger being at least one of a weakly acidic cation exchanger, a strongly acidic cation exchanger, or a mixture thereof.

According to a preferred embodiment, the method according to any of the preceding embodiments is characterized in that the strongly acidic cation exchanger is an organic, particularly preferably a synthetic resin ion exchanger, having sulfonic acid and/or sulfonate groups, preferably selected from a cross-linked polystyrene sulfonate or cross-linked poly(2-acrylamido-2-methyl-1-propanesulfonic acid) (polyAMPS).

According to a further embodiment, the method according to any one of the preceding embodiments is characterized in that the cation exchanger is partially or substantially completely loaded with H⁺ ions.

According to a further embodiment, the method according to any one of the preceding embodiments is characterized in that the ion exchanger is selected from the group consisting of anion exchangers, mixed anion and cation exchangers, cation exchangers loaded with transition metal ions, ion exchangers bearing chelate ligands, mixtures thereof and mixtures thereof with cation exchangers, wherein preferably the anion exchanger is partially or substantially completely loaded with OH⁻ ions.

According to a further embodiment, the method according to any one of the preceding embodiments is characterized in that the ion exchanger is at least one cation exchanger loaded with transition metal ions, wherein the transition metal ions are selected from the group consisting of cations of Ti, V, Cr, Mo, W, Mn, Fe, Ru, Co, Ni, Pd, Pt, Cu, Ag, Au, Zn, Cd, Hg, Pb, Ge, Sn, Al, or a lanthanide, and mixtures thereof, and preferably selected from the group consisting of cations of copper, silver, titanium, and mixtures thereof.

According to a further embodiment, the method according to any one of the preceding embodiments is characterized in that the ion exchanger is present in solid form, preferably as an organic ion exchanger and particularly preferably as a synthetic resin ion exchanger, wherein the ion exchanger may be present in particulate form, as a flow-through fill and/or as an insert in a mouth-nose protection.

According to a further embodiment, the method according to any one of the preceding embodiments is characterized in that the ion exchanger further comprises hygroscopic auxiliary agents and/or hygroscopic functional groups, or is used in combinations with such auxiliary agents.

According to a further embodiment, the method according to any one of the preceding embodiments is characterized in that the gases and/or gas streams are air and/or air streams, preferably selected from the group consisting of room air, room air streams and breathing air streams, wherein the room air and room air streams are preferably selected from room air and room air streams in shelters, motor vehicle interiors, air-conditioned rooms, cabins, airplanes, emergency vehicles, truck cabs, vehicles of security forces, respiratory equipment, intensive care units, staff rooms, rooms for animal husbandry and other rooms in which humans, animals or plants are present or are located.

According to a further embodiment, the method according to one of the preceding embodiments is characterized in that the ion exchanger is designed as a filling of a hollow body and/or as a porous molded body, the hollow body and/or the molded body preferably being inserted or incorporated into or being connected to a respiratory mask.

According to a further embodiment, the method according to any one of the preceding embodiments is carried out in combination with one or more of the methods selected from the group consisting of filtration, humidification, drying, condensation, UV treatment, corona or plasma treatment, high voltage treatment, radioactive radiation treatment, treatment by heating and treatment by cooling.

1. Use of one or more ion exchangers for the reduction and/or removal of biological contaminants in gases and/or gas streams.

2. The use according to claim 1, characterized in that the biological contaminants are selected from the group consisting of viruses, bacteria, molds, fungal spores, mites, mite residues, mite droppings, pollen, and fragments of the foregoing, metabolites such as mycotoxins, proteins, RNA and DNA, preferably selected from the group consisting of enveloped viruses, non-enveloped viruses, bacteria, fungal spores and proteins and particularly preferably selected from the group consisting of coronaviruses, SARS-type viruses, SARS-CoV-2 viruses, resistant pathogens and multi-resistant pathogens.

3. The use according to claim 1, characterized in that the ion exchanger comprises at least one cation exchanger, optionally the cation exchanger being at least one of a weakly acidic cation exchanger, a strongly acidic cation exchanger, or a mixture thereof.

4. The use according to claim 3, characterized in that the cation exchanger is partially or substantially completely loaded with H+ ions.

5. The use according to claim 1, characterized in that the ion exchanger is selected from the group consisting of anion exchangers, mixed anion and cation exchangers, cation exchangers loaded with transition metal ions, ion exchangers bearing chelate ligands, mixtures thereof and mixtures thereof with cation exchangers, wherein preferably the anion exchanger is partially or substantially completely loaded with OH− ions.

6. The use according to claim 5, characterized in that the ion exchanger is at least one cation exchanger loaded with transition metal ions, wherein the transition metal ions are selected from the group consisting of cations of Ti, V, Cr, Mo, W, Mn, Fe, Ru, Co, Ni, Pd, Pt, Cu, Ag, Au, Zn, Cd, Hg, Pb, Ge, Sn, Al, or a lanthanide, and mixtures thereof, and preferably are selected from the group consisting of cations of copper, silver, titanium and mixtures thereof.

7. The use according to claim 1, characterized in that the ion exchanger is present in solid form, preferably as an organic ion exchanger and more preferably as a synthetic resin ion exchanger, wherein the ion exchanger may be present in particulate form, as a flow-through fill and/or as an insert in a mouth-nose protection.

8. The use according to claim 1, characterized in that the ion exchanger further comprises hygroscopic auxiliary agents and/or hygroscopic functional groups.

9. The use according to claim 1, characterized in that the gases and/or gas streams are air and/or air streams, preferably selected from the group consisting of room air, room air streams and breathing air streams, wherein the room air and room air streams are preferably selected from room air and room air streams in shelters, motor vehicle interiors, air-conditioned rooms, cabins, airplanes, emergency vehicles, truck cabs, vehicles of security forces, respiratory equipment, intensive care units, staff rooms, rooms for animal husbandry and other rooms in which people, animals or plants are present or located.

10. The use according to claim 1, characterized in that the ion exchanger is designed as a filling of a hollow body and/or as a porous molded body, the hollow body and/or the molded body preferably being inserted or incorporated into or connected to a respiratory mask.

11. The use according to claim 1 in combination with one or more of the methods selected from the group consisting of filtration, humidification, drying, condensation, UV treatment, corona or plasma treatment, high voltage treatment, radioactive radiation treatment, treatment by heating and treatment by cooling.

12. Method for the removal and/or reduction of biological contaminants in gases and gas streams by means of one or more ion exchangers, characterized in that the gases or gas streams are brought into contact with an ion exchanger.

13. The method according to claim 12, characterized in that the biological contaminants are selected from the group consisting of viruses, bacteria, molds, fungal spores, mites, mite residues, mite droppings, pollen, and fragments of the foregoing, metabolites such as mycotoxins, proteins, RNA and DNA, preferably selected from the group consisting of enveloped viruses, non-enveloped viruses, bacteria, fungal spores and proteins and particularly preferably selected from the group consisting of coronaviruses, SARS-type viruses, SARS-CoV-2 viruses, resistant pathogens and multi-resistant pathogens.

14. The method according to claim 12, characterized in that the ion exchanger comprises at least one cation exchanger, optionally the cation exchanger being at least one of a weakly acidic cation exchanger, a strongly acidic cation exchanger, or a mixture thereof, and wherein the cation exchanger preferably is partially or substantially completely loaded with H+ ions.

15. The method according to claim 12, characterized in that the ion exchanger is selected from the group consisting of anion exchangers, mixed anion and cation exchangers and mixtures of anion and cation exchangers, wherein preferably the anion exchanger is partially or substantially completely loaded with OH− ions.

16. The method according to claim 12, characterized in that the ion exchanger is designed as a filling of a hollow body and/or as a porous molded body, preferably the hollow body and/or the molded body being inserted or incorporated into or connected to a respiratory mask.

17. The method according to claim 12, wherein additionally one or more methods selected from the group consisting of filtration, humidification, drying, condensation, UV treatment, corona or plasma treatment, high voltage treatment, radioactive radiation treatment, treatment by heating, treatment by cooling, ozonization, dosing of gases or liquids for treatment of the ion exchanger and/or the gas, in particular air, are carried out, wherein the method or methods are preferably carried out permanently or at intervals.
